(19) United States
(12) Patent Application Publication
(10) Pub. No.: US 2025/0177392 A1
(43) Pub. Date: Jun. 5, 2025

(54) BROAD SPECTRUM INHIBITORS OF CYTOMEGALOVIRUS

(71) Applicants: MICROBIOTIX, INC., Worcester, MA (US); ICAHN SCHOOL OF MEDICINE AT MOUNT SINAI, New York, NY (US)

(72) Inventors: Timothy J. Opperman, Arlington, MA (US); Domenico Tortorella, New York, NY (US); Steven M. Kwasny, Blackstone, MA (US); Zachary D. Aron, Acton, MA (US); Thomas Gardner, San Francisco, CA (US)

(73) Assignees: MICROBIOTIX, INC., Worcester, MA (US); ICAHN SCHOOL OF MEDICINE AT MOUNT SINAI, New York, NY (US)

(21) Appl. No.: 18/840,267

(22) PCT Filed: Feb. 22, 2023
(86) PCT No.: PCT/US2023/013580

§ 371 Date: Aug. 21, 2024

Related U.S. Application Data
(60) Provisional application No. 63/313,415, filed on Feb. 24, 2022.

(51) Int. Cl. A61K 31/506; A61K 31/5377; A61P 31/22

(52) U.S. Cl. CPC A61K 31/506; A61K 31/5377; A61P 31/22

(57) ABSTRACT

The present invention is related to the development of therapeutics and prophylactics for the treatment and/or prevention of cytomegalovirus infection in humans. A new class of N-arylpyrimidamine (NAPA) small molecules is disclosed that inhibits an early stage of the cytomegalovirus infection step of host cells. Also disclosed are methods of using the small molecule inhibitors in the treatment/prevention of cytomegalovirus infection.

### CROSS-REFERENCE TO RELATED APPLICATION

The presently disclosed subject matter claims the benefit of U.S. Provisional Patent Application Ser. No. 63/313,415 filed Feb. 24, 2022, the disclosure of which is incorporated herein by reference in its entirety.

### STATEMENT REGARDING FEDERALLY SPONSORED RESEARCH

This invention was made with government support under NIH grant AI113971 and AI139258. The United States Government has certain rights in the invention.

### FIELD OF THE INVENTION

This invention is in the field of therapeutic and prophylactic compounds to treat cytomegalovirus (CMV) infections and disease in human.

### BACKGROUND OF THE INVENTION

Human cytomegalovirus (CMV) is a genus of viruses in the order Herpesvirales, in the family Herpesviridae and is a ubiquitous pathogen with high seroprevalence rates (˜83%) worldwide (T. Adane and S. Getawa, J. Int. Med. Res., 49 (8): 3000605211034656 (2021)), and is known to infect mononuclear cells and lymphocytes. CMV can spread easily through bodily fluids such as blood, saliva, urine, semen, and breast milk but also from donor to recipient during organ transplants and across the placenta spreading from mother to fetus (Mocarski E S, Shenk T, Pass R F, Cytomegalovirus, pp. 2702-2772, In Knipe D M, Howley P M (ed), Fields Virology, 5th ed. Lippincott, Williams and Williams 2007). Virus proliferation significantly increases the morbidity and mortality of immunocompromised individuals, such as newborns, organ transplant recipients, AIDS patients, and the elderly. CMV is the leading cause of birth defects, affecting ˜0.5-2% of newborns worldwide with up to 40,000 new cases of CMV infection reported annually in the United States (Goderis et al., Pediatrics, 134:972-982 (2014); Ssentongo et al., JAMA Netw Open, 4 (8): e2120736 (2021); Damato E G and Winnen C W, J. Obstet. Gynecol. Neonatal. Nurs., 31:86-92 (2002)). CMV can also exacerbate cardiovascular diseases associated with atherosclerosis (Ji et al., Mol. Biol. Rep., 39:6537-6546 (2012); Lee et al., Biomarkers 19:109-13 (2014)) and cardiac allograft vasculopathy (Petrakopoulou et al., Circulation, 110 (11 Suppl 1): II207-12 (2004); Simmonds et al., Circulation, 117:2657-2661 (2008)). Given the large number of patient populations at risk for CMV-associated diseases and the estimated cost to treat CMV in the US ($4.4 billion/year by the National Academy of Sciences (Dove A., A long shot on cytomegalovirus, vol December, p 40-45. Richard Gallagher, Philadelphia, PA (2006). CMV is a significant health challenge requiring the development of a multi-faceted therapeutic strategy to limit CMV-associated diseases.

While several drugs have been approved by the FDA for the treatment of CMV infections, including formivirsen, ganciclovir (GCV), foscarnet (PFA), cidofovir (CDV) and letermovir (LTV), they exhibit high frequencies of drug resistance (Chou S., Rev. Med. Virol., 18:233-246 (2008); Jabs et al., J. Infect. Dis., 177:770-773 (1998); Weinberg et al., J. Infect. Dis., 187:777-784 (2003); Chou S., Antimicrob. Agents Chemother., 59:6588-6593 (2015)) and severe side effects (Kenneson A. and Cannon M. J., Rev. Med. Virol., 17:253-276 (2007)). All of these drugs target the late stage of viral replication and exhibit significant side effects and high resistance frequencies. GCV, PFA, and CDV are nucleoside analogs that inhibit viral DNA polymerase (UL54) (Lurain, N. S. and Chou, S., Clin. Microbiol. Rev., 23 (4): 689-712 (2010)). Unfortunately, the drugs impact all dividing cells leading to bone marrow toxicity and gastrointestinal disruption. In addition, PFA and CDV exhibit nephrotoxicity (Jabs et al., J. Infect. Dis., 177:770-773, (1998); Weinberg et al., J. Infect. Dis., 187:777-784 (2003); Chou et al., J. Infect. Dis., 176:786-789 (1997)). These significant adverse side effects can severely limit the use of these drugs in transplant recipients (Maffini et al., Expert Rev. Hematol., 9:585-596 (2016); Tan et al., Transpl. Infect. Dis., 16:556-560 (2014)). Moreover, mutations in the UL54 (polymerase) gene (Snydman et al., Transplant Proc., 43: S1-S17 (2011)) give rise to resistance at a frequency that can vary by transplanted organ [e.g. heart (0.3%), kidney (˜1%), kidney/pancreas (13%), lung (3-9%)] (Limaye A. P., Clin. Infect. Dis., 35:866-872 (2002)), contributing to treatment failure in 20-30% of transplant patients (Eid A. J. and Razonable, R. R., Drugs, 70:965-981 (2010)).

Letermovir (LTV) was approved by the FDA for a single indication only, prophylaxis of CMV infections in adult CMV-seropositive HSCT recipients (Chemaly et al., N. Engl. J. Med., 370:1781-1789 (2014); Marty et al., N. Engl. J. Med., 377:2433-2444 (2017)), which limits its clinical utility. LTV targets the viral terminase UL56, which inhibits packaging of the viral genome into mature virions (Goldner et al., J. Virol., 85:10884-10893 (2011)). LTV resistance was observed in one patient during the clinical trial (Marty et al., supra)) and LTV-resistance has emerged in subsequent use in humans (Cherrier et al., Am. J. Transplant, 18:3060-3064 (2018); Jung et al., BMC Infect. Dis., 19:388 (2019)). Therefore, it is likely that additional resistance will emerge in the clinical setting once the drug is used widely as a monotherapy. In addition, LTV also demonstrated increased cardiovascular adverse events and exhibits significant drug-drug interactions (e.g. statins) (Merck, Co. 2017. Prevymis package insert).

The development of alternative therapies is being pursued by numerous strategies through modification of current drugs, drug repurposing, and screening specific libraries. However, many new drugs targeting viral replication have failed to show efficacy in clinical trials. The replication inhibitor brincidofovir failed a phase 3 clinical trial in HSCT recipients (Marty et al., N. Engl. J. Med., 369:1227-1236 (2013)). Maribavir, an inhibitor of UL97 and viral DNA synthesis (Biron et al., Antimicrob. Agents Chemother., 46:2365-2372 (2002)) failed a phase 3 trial of 681 HSCT patients (Marty et al., Lancet Infect. Dis., 11:284-292 (2011)) and is currently undergoing a phase 2 trial with higher doses (Maffini et al., Expert Rev. Hematol., 9:585-596 (2016)). Also, maribavir treatment appears to rapidly induce resistant strains and CMV naturally expresses a resistant isoform of UL97 (Webel et al., J. Virol., 88:4776-4785 (2014)). Leflunomide, a rheumatoid arthritis drug that prevents the maturation of virions (Waldman et al., Intervirology, 42:412-418 (1999)) gave inconsistent results on its efficacy for transplant recipients (Avery et al., Transplantation, 90:419-426 (2010); Chacko, B. and John, G. T., Transpl. Infect. Dis., 14:111-120 (2012); Battiwalla et al., Transpl. Infect. Dis., 9:28-32 (2007)). Additionally, artemisinins have anti-CMV properties (Arav-Boger et al., PLOS One, 5: e10370 (2010)) by targeting the cell cycle, but mixed results were observed in its effectiveness in HSCT recipients (Germi et al., Antiviral Res., 101:57-61 (2014)). The limited effectiveness of these late-phase inhibitors exemplifies the need for novel CMV therapeutics that target earlier stages of the viral life cycle.

Therefore, in view of the lack of HCMV vaccines, there is currently a significant need worldwide for a vaccine that is safe and effective in all patient populations to prevent and/or treat CMV infection.

### SUMMARY OF THE INVENTION

The present invention is directed to the identification, isolation, and characterization of small molecule inhibitors of cytomegalovirus (CMV) for use in the treatment and/or prevention of CMV infections in mammals, and in particular the treatment and/or prevention of human cytomegalovirus (HCMV) infections in humans. It is envisioned that these novel inhibitors will be especially beneficial in the treatment or prevention of HCMV infection in immunocompromised or immunosuppressed patients which are at a higher risk for infection.

More specifically, the present invention is directed to a novel class of compounds that inhibit CMV infection by advantageously targeting the early stages of CMV replication. This novel class of compounds referred to herein as N-arylpyrimidinamines (NAPA), demonstrated potent anti-CMV activity in low μM levels with a high selectivity index of >30 and favorable in vitro ADME properties. Mechanism of action studies demonstrated that the NAPA compounds specifically inhibit CMV at an early stage of its lifecycle. A potent NAPA analog was evaluated against a panel of herpes viruses and was active against human CMV at a potency comparable to ganciclovir but also provided protection in a ganciclovir-resistant CMV strain. Further, a number of NAPA analogs demonstrated high potency to inhibit CMV infection and reduced dissemination of diverse strains in physiological relevant cell types. Importantly, combination drug studies utilizing NAPA compounds with ganciclovir demonstrated a more potent synergistic inhibitory impact on CMV dissemination in physiologically important cell types.

Therefore, in one aspect, the present invention is directed to the discovery of a novel broad-spectrum small molecule class of N-arylpyrimidinamine (NAPA) compounds as inhibitors of cytomegalovirus. Advantageously, the novel NAPA compounds described herein prevent entry of CMV into a host cell. The novel NAPA inhibitor compounds described herein are suitable for the treatment and/or prevention of CMV infections in mammals. More particularly, the novel NAPA inhibitor compounds described herein are suitable for the treatment and/or prevention of CMV infections in humans.

As described below, using standard assays, the novel NAPA compounds of the present invention demonstrated the ability to effectively prevent cytomegalovirus infection of both human fibroblasts and epithelial cells with IC₅₀ values in the very low (≤5 μM) range.

In one embodiment, the present invention is directed to a novel CMV inhibitor compound having the structure of Formula I:

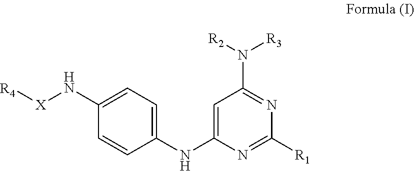

wherein:
R₁ is selected from a hydrogen atom, or a C1-C3 alkyl group;
R₂ and R₃ are selected from a hydrogen atom, a C1-C3 alkyl group, an aromatic ring with 1-3 substituents selected from a halogen, a C1-C4 alkyl group, a C1-C4 alkoxy group, or a C1-C4 aminoalkyl group, a heteroaromatic ring with 1-3 substituents selected from a halogen, C1-C4 alkyl group, a C1-C4 alkoxy group, or a C1-C4 amino-alkyl group,
R₂ and R₃ may be a bridging chain to form a heterocyclic ring with 1-3 substituents selected from a C1-C4 alkyl group, a C1-C4 alkoxy group, or a C1-C4 aminoalkyl group,
R₄ is a substituted or non-substituted furan, thiophene, or phenyl ring; and
X is a carbonyl (CO) group or a sulfonyl (SO₂) group;
or a pharmaceutically acceptable salt thereof.

In another embodiment, the present invention is directed to a compound of Formula I for use as an inhibitor of cytomegalovirus and in particular an inhibitor of human cytomegalovirus.

In another embodiment, the present invention is directed to a compound for use in a method of treating or preventing a cytomegalovirus infection in a mammalian subject, the method comprising administering to a subject in need thereof an effective amount of a cytomegalovirus inhibitor compound having the structure of Formula I.

In another embodiment, the present invention is directed to a composition comprising at least one compound for use in a method of inhibiting cytomegalovirus infection in a mammal, the method comprising administering an effective amount of a composition comprising at least one compound, the compound having the structure of Formula I.

In another embodiment, the present invention is directed to a pharmaceutical composition comprising at least one cytomegalovirus inhibitor compound of Formula I, and a pharmaceutically acceptable carrier or excipient.

In another embodiment, the present invention is directed to a pharmaceutical composition for use in the treatment or prevention of a cytomegalovirus infection, the composition comprising a compound of Formula I.

In another embodiment the present invention is directed to the use of a compound of Formula I in the manufacture of a medicament for use the treatment or prevention of a cytomegalovirus infection.

In another embodiment, the novel CMV inhibitor compounds of the present invention comprise a compound having the structure of Formula II:

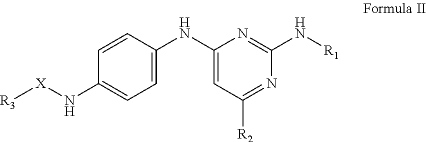

wherein:

R₁ is a substituted phenyl ring with 1-3 substituents selected from a halogen, C1-C4 alkyl group, a C1-C4 alkoxy group, or a C1-C4 amino-alkyl group;

R₂ is a C1-C3 alkyl group;

R₃ is a substituted phenyl or furan ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and

X is a carbonyl (CO) group, a sulfonyl (SO₂) group, or a NHCO group (part of a urea).

In another embodiment, the present invention is directed to a compound of Formula II for use as an inhibitor of cytomegalovirus and in particular an inhibitor of human cytomegalovirus.

In another embodiment, the present invention is directed to a compound for use in a method of treating or preventing a cytomegalovirus infection in a mammalian subject, the method comprising administering to a subject in need thereof an effective amount of a cytomegalovirus inhibitor compound having the structure of Formula II.

In another embodiment, the present invention is directed to a composition comprising at least one compound for use in a method of inhibiting cytomegalovirus infection in a mammal, the method comprising administering an effective amount of a composition comprising at least one compound, the compound having the structure of Formula II.

In another embodiment, the present invention is directed to a pharmaceutical composition comprising at least one cytomegalovirus inhibitor compound of Formula II, and a pharmaceutically acceptable carrier or excipient.

In another embodiment, the present invention is directed to a pharmaceutical composition for use in the treatment or prevention of a cytomegalovirus infection, the composition comprising a compound of Formula II.

In another embodiment the present invention is directed to the use of a compound of Formula II in the manufacture of a medicament for use the treatment or prevention of a cytomegalovirus infection.

In another embodiment, the novel CMV inhibitor compounds of the present invention comprise a compound having the structure of Formula III:

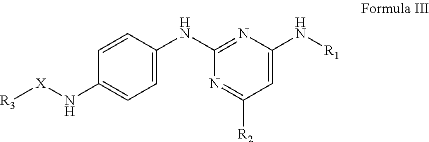

wherein:
R₁ is a phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is substituted phenyl ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is a carbonyl (CO) group.

In another embodiment, the present invention is directed to a compound of Formula III for use as an inhibitor of cytomegalovirus and in particular an inhibitor of human cytomegalovirus.

In another embodiment, the present invention is directed to a compound for use in a method of treating or preventing a cytomegalovirus infection in a mammalian subject, the method comprising administering to a subject in need thereof an effective amount of a cytomegalovirus inhibitor compound having the structure of Formula III.

In another embodiment, the present invention is directed to a composition comprising at least one compound for use in a method of inhibiting cytomegalovirus infection in a mammal, the method comprising administering an effective amount of a composition comprising at least one compound, the compound having the structure of Formula III.

In another embodiment, the present invention is directed to a pharmaceutical composition comprising at least one cytomegalovirus inhibitor compound of Formula III, and a pharmaceutically acceptable carrier or excipient.

In another embodiment, the present invention is directed to a pharmaceutical composition for use in the treatment or prevention of a cytomegalovirus infection, the composition comprising a compound of Formula III.

In another embodiment the present invention is directed to the use of a compound of Formula III in the manufacture of a medicament for use the treatment or prevention of a cytomegalovirus infection.

Specific NAPA CMV inhibitor compounds of the present invention are selected from the group consisting of:

| Cmpd # | Structure |
|---|---|
| MBXC- 4294 | 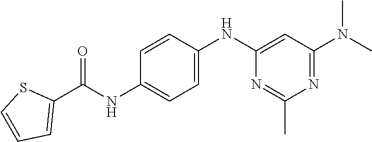 [SMILES: Cc1nc(Nc2ccc(NC(=O)c3cccs3)cc2)cc(N(C)C)n1] |
| MBXC- 4295 | 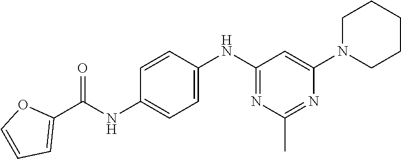 [SMILES: Cc1nc(Nc2ccc(NC(=O)c3ccco3)cc2)cc(N2CCCCC2)n1] |
| MBXC- 4297 | 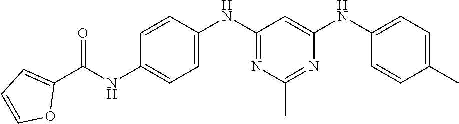 [SMILES: Cc1ccc(Nc2cc(Nc3ccc(NC(=O)c4ccco4)cc3)nc(C)n2)cc1] |
| MBXC- 4298 | 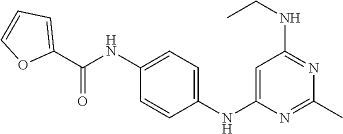 [SMILES: CCNc1cc(Nc2ccc(NC(=O)c3ccco3)cc2)nc(C)n1] |
| MBXC- 4299 | 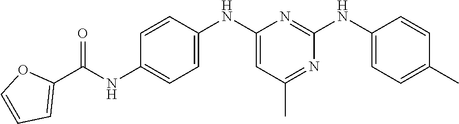 [SMILES: Cc1ccc(Nc2nc(C)cc(Nc3ccc(NC(=O)c4ccco4)cc3)n2)cc1] |
| MBXC- 4300 | 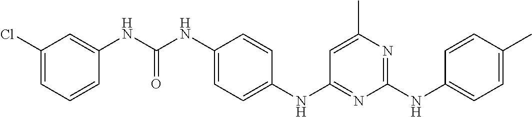 [SMILES: Cc1ccc(Nc2nc(C)cc(Nc3ccc(NC(=O)Nc4cccc(Cl)c4)cc3)n2)cc1] |
| MBXC- 4301 | 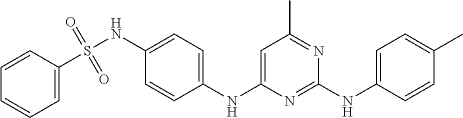 [SMILES: Cc1ccc(Nc2nc(C)cc(Nc3ccc(NS(=O)(=O)c4ccccc4)cc3)n2)cc1] |
| MBXC- 4302 | 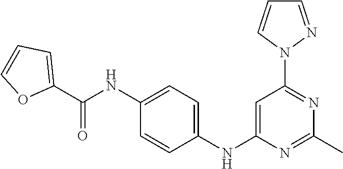 [SMILES: Cc1nc(Nc2ccc(NC(=O)c3ccco3)cc2)cc(-n2cccn2)n1] |
| MBXC- 4303 | 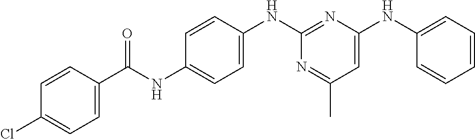 [SMILES: Cc1cc(Nc2ccccc2)nc(Nc2ccc(NC(=O)c3ccc(Cl)cc3)cc2)n1] |
| MBXC- 4304 | 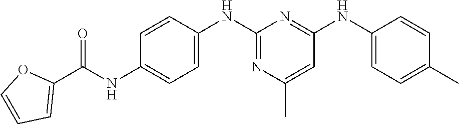 [SMILES: Cc1ccc(Nc2cc(C)nc(Nc3ccc(NC(=O)c4ccco4)cc3)n2)cc1] |
| MBXC- 4325 | 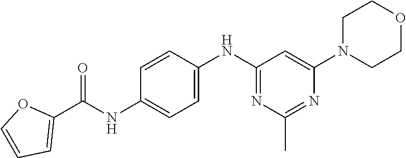 [SMILES: Cc1nc(Nc2ccc(NC(=O)c3ccco3)cc2)cc(N2CCOCC2)n1] |
| MBXC- 4330 | 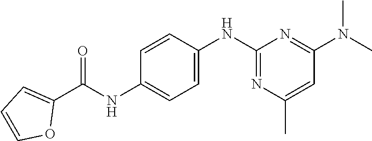 [SMILES: Cc1cc(N(C)C)nc(Nc2ccc(NC(=O)c3ccco3)cc2)n1] |
| MBXC- 4336 | 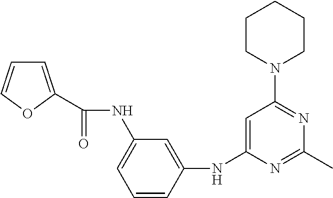 [SMILES: Cc1nc(Nc2cccc(NC(=O)c3ccco3)c2)cc(N2CCCCC2)n1] |
| MBX- 4991 | 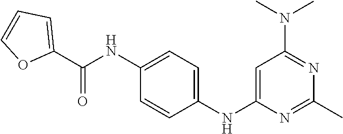 [SMILES: Cc1nc(Nc2ccc(NC(=O)c3ccco3)cc2)cc(N(C)C)n1] |
| MBX- 4992 | 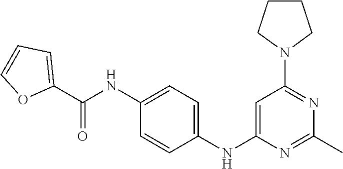 [SMILES: Cc1nc(Nc2ccc(NC(=O)c3ccco3)cc2)cc(N2CCCC2)n1] |

In another embodiment, the present invention is directed to a composition for treating or preventing CMV infection, the composition comprising a novel NAPA CMV inhibitor compound according to Formula I, II, and/or III described herein. The compositions described herein are suitable for the treatment and/or prevention of CMV infections in mammals, and in particular, the treatment and/or prevention of CMV in humans.

In another embodiment, the present invention is directed to a method for treating or preventing CMV infections in a mammal by administration of the novel NAPA CMV inhibitor compounds of Formula I, II, and/or III. In a preferred embodiment, the mammal is a human.

In another embodiment, the present invention is directed to the use of the novel NAPA CMV inhibitor compounds of Formula I, II, and/or III, or a salt thereof, to inhibit infection of a mammalian host cell by CMV. In a preferred embodiment, the mammal is a human.

The present invention is further directed to the use of the novel NAPA compounds described herein in a method for the manufacture of a medicament for treating or preventing CMV infection in mammals (e.g., humans) comprising combining one or more of the NAPA CMV inhibitor compounds of Formula I, Formula II, and/or Formula III of the present invention, products, or compositions with a pharmaceutically acceptable carrier or diluent.

In another aspect, the present invention is directed to pharmaceutical compositions comprising a therapeutically effective amount of a novel NAPA CMV inhibitor compound of the present invention, or a pharmaceutically acceptable salt thereof, and a pharmaceutically acceptable carrier. The pharmaceutical compositions are suitable for use in the disclosed methods for treating or preventing CMV infections in a mammal. The pharmaceutical compositions may be formulated for both parenteral and/or nonparenteral administration to a subject or patient in need thereof.

In another embodiment, the novel NAPA inhibitors of the present invention may be administered to a subject in need thereof optionally in combination with one or more additional antiviral agent or agents. The additional agent or agents may be combined with the novel NAPA compounds of the present invention to create a single pharmaceutical dosage form. Alternatively, these additional agents may be separately administered to the patient as part of a multiple dosage, for example using a kit. Such additional agents may be administered to the patient prior to, concurrently with, or following the administration of the novel NAPA compounds described herein, or a pharmaceutically acceptable salt thereof. By way of example, the second agent or agents may be selected from, but not limited to, ganciclovir, foscarnet, cidofovir, maribavir, and valganciclovir.

In another embodiment, the NAPA CMV inhibitors of the present invention are formulated into a pharmaceutically acceptable carrier and are applied/administered to a subject in need thereof by an injection, including, without limitation, intradermal, transdermal, intramuscular, intraperitoneal, and intravenous. According to another embodiment of the invention, the administration is oral and the compound may be presented, for example, in the form of a tablet or encased in a gelatin capsule or a microcapsule, which simplifies oral application. The production of these forms of administration is within the general knowledge of a technical expert. Multiple routes of administration are envisioned for these drug-like molecules, and highly cost-effective production strategies can be easily achieved.

In a preferred embodiment, the CMV inhibitors of the present invention will specifically target and inhibit an early stage of the CMV lifecycle shortly after, preferably within 0 to three hours of viral attachment (infection) to a host cell.

In preferred embodiments, the novel NAPA compounds of the present invention exhibit potent antiviral activity against CMV strains (IC₅₀≤10 μM), favorable cytotoxicity, i.e., CC₅₀≥100 μM, optimal in vivo drug interaction, i.e., a minimal inhibition of CYP450 (<30% at 10 μM), favorable bioavailability, i.e., a Caco-2 permeability value (Pₐₚₚ) of >1×10⁻⁶ Cm/sec, and a selectivity index (CC₅₀/IC₅₀)≥100.

As described below, to identify inhibitors that prevent entry of CMV into host cells, we developed a high-content screening (HCS) assay using a CMV AD169 yellow-fluorescent protein (YFP) expressing virus (39) to screen >112,000 compounds to discover compounds that target an early step of virus entry.

### Definitions

A composition or method described herein as “comprising” (or “comprises”) one or more named elements or steps is open-ended, meaning that the named elements or steps are essential, but other elements or steps may be added within the scope of the composition or method. To avoid prolixity, it is also understood that any composition or method described as “comprising” one or more named elements or steps also describes the corresponding, more limited, composition or method “consisting essentially of” (or “consists essentially of”) the same named elements or steps, meaning that the composition or method includes the named essential elements and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other element or step. In any composition or method disclosed herein, known or disclosed equivalents of any named essential element or step may be substituted for that element or step, respectively.

As used herein, the term “subject” can be a human, non-human primate, horse, pig, rabbit, dog, sheep, goat, cow, cat, guinea pig or rodent. A “patient” or “subject in need thereof” refers to a mammal afflicted with a disease or disorder. The term “patient” includes human and veterinary subjects.

Terms such as “parenteral”, “parenterally”, and the like, refer to routes or modes of administration of a compound or composition to an individual other than along the alimentary canal. Examples of parenteral routes of administration include, without limitation, subcutaneous (s.c.), intravenous (i.v.), intramuscular (i.m.), intra-arterial (i.a.), intraperitoneal (i.p.), transdermal (absorption through the skin or dermal layers), nasal (“intranasal”; absorption across nasal mucosa), or pulmonary (e.g., inhalation for absorption across the lung tissue), vaginal, direct injections or infusions into body cavities or organs other than those of the alimentary canal, as well as by implantation of any of a variety of devices into the body (e.g., of a composition, depot, or device that permits active or passive release of a compound or composition into the body).

The terms “non-parenteral”, “non-parenterally”, “enteral”, “enterally”, “oral”, “orally”, and the like, refer to administration of a compound or composition to an individual by a route or mode along the alimentary canal. Examples of enteral routes of administration include, without limitation, oral, as in swallowing solid (e.g., tablet) or liquid (e.g., syrup) dosage forms, sublingual (absorption through the mucosal membranes lining the floor of the mouth, e.g., under the tongue), buccal (absorption through the mucosal membranes lining the cheeks), nasojejunal or gastrostomy tubes (delivery into the stomach), intraduodenal administration, as well as rectal administration (e.g., suppositories for release of a drug composition into and absorption by the lower intestinal tract of the alimentary canal).

In the present description, in a structural formula allowing for one or more substituent at a given position and listing suitable substituents, it will be understood that substituents may be “stacked” or combined to form compound substituents. For example, a listing of suitable substituents including alkyl and aryl substituents, aralkyl and alkaryl substituents are also contemplated.

An “effective amount” or a “therapeutically effective amount” of a compound is that amount necessary or sufficient to treat or prevent a CMV infection as described herein. In an example, an effective amount of a NAPA CMV inhibitor described herein is an amount sufficient to treat or prevent a CMV infection in a mammalian subject, preferably a human. The effective amount or therapeutically effective amount will vary depending on the cell, the severity and prevalence of the disease, and the age, weight, etc. of the subject to be treated.

The term “coadministration” and “in combination with” include the administration of a novel NAPA compound described herein in combination with at least one or more additional therapeutic agent or agents administered either simultaneously, concurrently, or sequentially within no specific time limits. In one embodiment, the therapeutic agent or agents are in the same composition as the NAPA inhibitor compound in unit dosage form or alternatively are in separate compositions from the NAPA inhibitor compound in unit dosage form.

The term “unit dosage form” as used herein, refers to physically discrete units suitable as unitary dosages for treatment or prevention of a CMV infection, each unit containing a predetermined quantity of an active agent, calculated in an amount sufficient to produce the desired effect in association with a pharmaceutically acceptable diluent, carrier, or vehicle.

The term “hydrogen” is intended to mean a hydrogen radical.

The term “alkyl” as used herein is intended to mean a branched, straight-chain, or cyclic saturated hydrocarbon group of 1 to 24 carbon atoms, preferably 1-10 carbon atoms such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, and the like. The alkyl group can be cyclic or acyclic. The alkyl group can be branched or unbranched. The alkyl group can also be substituted or unsubstituted. For example, the “substituted alkyl” group denotes an alkyl group substituted with one or more groups including, but not limited to, alkyl, haloalkyl, nitro, halogen, alkoxy, alkylthio, haloalkoxy, sulfonyl, sulfinyl, carboxy, alkoxycarbonyl, or amido groups.

The term “alkoxy” is intended to mean the radical-OR, where R is an alkyl or cycloalkyl group.

The term “sulfonyl” is intended to mean a sulfur radical that is doubly bound to two oxygens (—SO₂—). A sulfonyl group may be linked via the sulfur atom with an amino, alkylamino, alkyl, cycloalkyl, aryl, heterocycloalkyl, or heteroaryl moiety to produce a monovalent radical.

The term “amino” is intended to mean the radical —NH₂.

The term “halo” or “halogen” means fluorine, chlorine, bromine, or iodine.

The term “furan” refers to a heterocyclic compound consisting of a five-membered aromatic ring with four carbon atoms and one oxygen atom.

The term “thiophene” refers to a heterocyclic compound having the formula C₄H₄S.

The term “urea” refers to a compound having the formula CO(NH₂)₂.

### BRIEF DESCRIPTION OF THE DRAWINGS

FIG. 1 shows the structure of NAPA compound MBXC-4302 with anti-CMV activity (IC₅₀) and cytotoxicity (CC₅₀) values.

FIG. 2A-D are graphs showing the inhibitory effect of MBXC-4302 against HCMV infection. HCMV strains AD169^{R} (A and C) and TB40/E (B and D) infection (MOI 0.25) of fibroblasts (A and B) and ARPE-19 cells (C and D) pre-treated with 0, 1, 5, and 20 μM MBXC-4302 were assessed 24 hpi based on GFP fluorescence (A and C) or IE1 (B and D) expression by an anti-IE1 immunostain. The % infection was determined using DMSO-treated as 100% infection. The error bars are based samples in triplicate. Statistical tests were performed using ordinary one-way ANOVA with multiple comparisons to DMSO treated cells as a control and a Dunnett's post-test; ****, p<0.0001.

FIG. 3 shows the results of the structure activity relationship HCMV inhibitory studies of the NAPA analogs.

FIG. 4A-E are graphs showing that MBX-4992 significantly limits CMV infection in vitro. HCMV strains AD169^{R} (A and C) and TB40/E (B and D) infection (MOI 0.2 or 0.4, respectively) of fibroblasts (A and B) and ARPE-19 cells (C and D) pre-treated with 0, 1, 5, and 20 μM MBX-4992 were assessed 24 hpi based on GFP fluorescence (A and C) or IE1 (B and D) expression by anti-IE1 immunostain. The % infection was determined using DMSO-treated as 100% infection. The error bars are based on samples carried out in triplicate. Statistical tests were performed using ordinary one-way ANOVA with multiple comparisons to DMSO treated cells as a control and a Dunnett's post-test; ***, p<0.001; ****, p<0.0001.

FIG. 4E shows total cell lysates from AD169^{R}-infected (MOI 0.2) NHDF cells pre-treated with DMSO, MBX-4992 (1-10 μM), ganciclovir (5 μM), or convallotoxin (0.01 μM) collected at 24 hpi were subjected to Western blot analysis using a rabbit anti-IE-1 antibody (Lanes 1-6) and an anti-GAPDH antibody (Lanes 7-12) as a loading control. The molecular weight markers and respective proteins are indicated.

FIG. 5A-C are graphs showing that NAPA compounds target an early, post-attachment step of the CMV virus. (A) MBX-4992 (10 μM) was added along a time course post infection with AD169 (MOI 0.2) from −60 to 90 minutes after virus addition to NHDF fibroblasts. Infection at 24 hours was quantified after an immunostain with an anti-IE1 antibody. Percent infection was normalized to untreated cells where media was added either-60 or 90 minutes after virus addition. A schematic of the experimental design is shown.

(B) Based on the schematic, AD169^{R} (MOI 0.2) was pre-incubated with DMSO, MBX-4992, or heparin for one hour before being added to NHDF cells. Virus/compound mix was washed out after 30 minutes as indicated and either unchanged or replaced with media containing DMSO, MBX-4992, or heparin. Virus infection was quantified at 24 hpi GFP fluorescence. Percent infection was normalized to untreated cells.

(C) NHDF cells were pre-incubated with DMSO, MBX-4992 or heparin prior to virus addition. After addition of AD169^{R} (MOI 0.2) for 30 min, media was either unchanged or replaced with media containing DMSO, MBX-4992, or heparin. Virus infection was quantified at 24 hpi by GFP fluorescence. Percent infection was normalized to untreated cells. Statistical tests were performed using ordinary one-way ANOVA with multiple comparisons to DMSO treated cells as a control and a Dunnett's post-test: *, p<0.05; ****, p<0.0001.

FIG. 6A-D are graphs showing that NAPA analogs effectively prevent a CMV infection. HCMV strain AD169 infection (MOI 0.2) of NHDF cells treated with NAPA compounds MBX-4992 (A), MBXC-4325 (B), MBXC-4330 (C), and MBXC-4336 (D) were analyzed 24 hpi by immunostaining for IE1 protein levels. The % infection was determined using DMSO-treated as 100% infection. The error bars are based on samples done in triplicate. Statistical tests were performed using ordinary one-way ANOVA with multiple comparisons to DMSO treated cells as a control and a Dunnett's post-test; *, p<0.05; **, p<0.01; ****, p<0.0001. The chemical structure for each inhibitor compound is shown below results for each neutralization assay.

FIG. 7A-D are graphs showing that NAPA compounds exhibit favorable cytotoxicity and cytostaticity profiles. Fibroblasts were treated with increasing concentrations of MBX-4992 and MBXC-4336 for up to 6 days and cell number was measured periodically using Hoechst stain to quantify cell proliferation (A and B). Cyclohexamide (CHX) and DMSO (−) were used as positive and negative controls for cytotoxicity respectively. Cell cytostaticity (C and D) was assessed using ATP-based CellTiter Glo luciferase assay following extended incubation with increasing concentrations of MBX-4992 and MBXC-4336 for 1 or 6 days. Relative light units (RLU) were measured following cell lysis and normalized to DMSO treated cells for each time point.

FIG. 8A-H are fluorescence images (A & B) and graphs showing that NAPA compounds MBX-4992 and MBXC-4336 work in both prophylactic and therapeutic settings to inhibit HCMV infection. ARPE-19 cells (A to D) and NHDF cells (E and F) treated with increasing concentrations (0, 1, 5, 20, and 50 μM) of MBX-4992, MBXC-4336, or ganciclovir (2.5 μM) were infected with AD169^{R} (MOI 0.025) and analyzed for viral plaques of >5,000 μm² (C and E) and >10,000 μm² (D and F) based on GFP fluorescence. The representative images of plaques from AD169^{R}-infected ARPE-19 cells treated MBX-4992, MBXC-4336, ganciclovir are shown (A and B). MBX-4992, MBXC-4336 (G and H), and ganciclovir treatment at 48 hpi of AD169^{R}-infected ARPE-19 cells (MOI 0.025) were analyzed for virus plaques >5,000 μm² and >10,000 μm² at 10 dpi. The normalized plaque number was designated to be 100 based on plaques from DMSO treated cells. Statistical tests were performed using ordinary one-way ANOVA with multiple comparisons to DMSO treated cells as a control and a Dunnett's post-test; *, p<0.05; **, p<0.01; ***, p<0.001; ****, p<0.0001.

FIG. 9A-D are fluorescence images and graphs showing that MBX-4992 in combination with ganciclovir is effective to inhibit CMV infection. ARPE-19 (A and C) and NHDF (B and D) cells infected with TB40/E (MOI 0.002) were treated with DMSO, MBX-4992 (0, 1, 5, or 20 μM), and ganciclovir (2.5 μM). At 10 day post-infection, representative whole well images of plaques >10,000 μm² (A and B) were quantified based on GFP fluorescence intensity and calculated relative to DMSO controls (C and D). Statistical tests were performed using ordinary one-way ANOVA with multiple comparisons to DMSO treated cells as a control and a Dunnett's post-test. *, p<0.05; **, p<0.01; ***, p<0.001; ****, p<0.0001.

FIG. 10A-C shows that MBX-4992 prevents HCMV infection. HCMV strains AD169^{R} (A) and AD169 (B) infection (MOI 0.2) of NHDF cells pre-treated with 0, 5, and 20 μM MBX-4992 were assessed at 1 dpi or 2 dpi based on IE1 expression by anti-IE1 immunostain. The % infection was determined using DMSO-treated as 100% infection. The error bars represent values in triplicate. Statistical tests were performed using ordinary one-way ANOVA with multiple comparisons to DMSO treated cells as a control and a Dunnett's post-test; **, p<0.01; ****, p<0.0001. (C) Total cell lysates from AD169^{R}-infected (MOI 0.2) ARPE-19 cells pre-treatment with DMSO, MBX-4992 (1-20 μM) or heparin (50 μg/mL) collected at 24 hpi were subjected to Western blot analysis using a rabbit anti-IE-1 antibody (lanes 1-7) and an anti-GAPDH antibody (lanes 8-14) as a loading control. The molecular weight markers and respective proteins are indicated.

FIG. 11A-J are graphs showing that MBX-4992 exhibits broad inhibition against a number of HCMV strains in various cell types. HCMV strains AD169 (A), TB40/E Flag YFP (B), Towne (C and G), TR (D and F), Merlin (E), and TB40/E (H) infection of NHDF cells pre-treated with DMSO and 0, 1, 5, or 20 μM MBX-4992 or MBXC-4336, respectively, were assessed at 24 hpi based on IE1 expression by anti-IE1 immunostain. TB40/E (I) (MOI 0.25) and AD169^{R} (J) (MOI 0.25) infection of HTR-svNeo cells treated with DMSO and 0, 1, 5, or 20 μM MBX-4992 were assessed at 24 hpi based on IE1 expression by anti-IE1 immunostain. The % infection was determined using DMSO-treated as 100% infection. The error bars represent values in triplicate. Statistical tests were performed using ordinary one-way ANOVA with multiple comparisons to DMSO treated cells as a control and a Dunnett's post-test *, p<0.05; **, p<0.01; ****, p<0.0001.

FIGS. 12A and B are Western Blot assays showing that MBXC-4336 limits expression of IE1 in fibroblasts and epithelial cells. Total cell lysates from AD169^{R}-infected (MOI 0.2) NHDF (A) and ARPE-19 (B) cells pre-treated with DMSO, MBX-4336 (0, 1, 5, 10, or 20 μM), or heparin (50 μg/mL) collected at 48 hpi were subjected to Western blot analysis using a rabbit anti-IE-1 antibody (lanes 1-7) and an anti-GAPDH antibody (lanes 8-14) as a loading control. The molecular weight markers and respective proteins are indicated.

FIGS. 13A and B are graphs showing that MBX-4992 limits the generation of infectious virions in fibroblasts and epithelial cells. MRC5 and ARPE-19 cells infected with AD169^{R} (MOI 0.5) were treated with DMSO, MBX-4992 (1, 5, 10, and 20 μM), and ganciclovir (5 μM) for 5 days, washed, and replaced with media for 3 days. The supernatant of MRC5 treated-cells (A) and ARPE-19-treated (B) cells was used to infect MRC5 cells. Virus infection was analyzed 24 hpi using GFP fluorescent signal as the readout with a Celigo cytometer. The viral infectious dose (IU/ml) was determined from each condition. The error bars are based on differences of infectious dose from 5 samples. Statistical tests were performed using ordinary one-way ANOVA with multiple comparisons to DMSO treated cells as a control and a Dunnett's post-test ****, p<0.0001.

### DETAILED DESCRIPTION OF THE INVENTION

The present invention is directed to the discovery, isolation, and characterization of novel compounds and methods for treating and/or preventing a cytomegalovirus (CMV) infection in a mammalian subject. The CMV life cycle (˜96 hrs) is a complex process requiring both cellular and viral factors initiated with virus binding to the cell surface followed by a fusion event releasing the viral capsid into the cytosol where it traffics to the nucleus initiating viral gene expression. The viral genome is then replicated then packaged into the capsid in the nucleus followed by trafficking through the cell with a final envelopment in the Golgi apparatus and release from the cell. The novel compounds described herein preferably act by preventing entry of the CMV into a host cell.

Without in any way limiting the present invention, it is believed the novel CMV inhibitor compounds described herein are likely targeting a post-attachment step of CMV infection and are likely targeting the gB protein-mediated fusion step of the CMV infection cycle. It is believed the novel compounds described herein are unique as they are the first to target this particular step in the CMV infection pathway. In addition, the inhibitor compounds appear to be quite specific for cytomegalovirus replication based the IC50 values for human CMV (˜1.8 mM) and mouse CMV (4.5 mM).

To identify CMV inhibitors, a high-content screening (HCS) early-stage-specific reporter assay using a CMV AD169 yellow-fluorescent protein (YFP) expressing virus (AD169^{IE2-YFP}) (Gardner et al., Antiviral Res., 113:49-61 (2015)) was developed to screen >112,000 compounds to identify potent inhibitors (IC50<10 μM) of cytomegalovirus having a minimal mammalian cytotoxicity (CC₅₀) of preferably ≥100 μM. As a result, we have identified, isolated, and characterized a novel series of N-arylpyrimidinamines (NAPAs) that inhibit CMV infection. The compounds exhibit advantageous drug-like properties and a responsive SAR, suggesting the novel NAPA compounds act on a discreet viral or host target. Further, the NAPA compounds are broadly effective at inhibiting virus infection of diverse strains and cell types. Further, the NAPA compounds limit proliferation and production in both fibroblasts and epithelial cells demonstrating its effectiveness for both prophylactic and therapeutic applications.

This NAPA series is highly attractive for drug development, and they appear to act by specifically blocking or disrupting the crucial CMV/host cell interaction required for virus entry into a host cell. Members of the NAPA series were highly effective against CMV with concentration-dependent decreases in infection and IC₅₀ values as low as 1.7 μM, which is comparable to the known viral inhibitor, ganciclovir. In addition, the novel NAPA compounds of the present invention exhibited broad spectrum inhibition of various CMV strains including AD169, Towne, TB/40E, and Merlin. Therefore, the NAPA series provides broad-spectrum protection against CMV infection.

Human cytomegalovirus infection causes significant morbidity and mortality in immunocompromised individuals, including organ transplant recipients, AIDS patients, newborns, cancer patients, autoimmune patients, and the elderly. Advantageously, the novel compounds described herein, when administered to such immunocompromised patients will limit viral proliferation and significantly reduce cytomegalovirus-associated diseases and mortality.

Therefore, in one embodiment, the novel CMV inhibitor compounds of the present invention comprise a compound having the structure of Formula I:

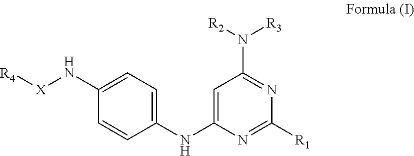

wherein:
R₁ is selected from a hydrogen atom, or a C1-C3 alkyl group; R₂ and R₃ are selected from a hydrogen atom, a C1-C3 alkyl group, an aromatic ring with 1-3 substituents selected from a halogen, a C1-C4 alkyl group, a C1-C4 alkoxy group, or a C1-C4 amino-alkyl group, a heteroaromatic ring with 1-3 substituents selected from a halogen, C1-C4 alkyl group, a C1-C4 alkoxy group, or a C1-C4 amino-alkyl group,
R₂ and R₃ may be a bridging chain to form a heterocyclic ring with 1-3 substituents selected from a C1-C4 alkyl group, a C1-C4 alkoxy group, or a C1-C4 amino-alkyl group,
R₄ is a substituted or non-substituted furan, thiophene, or phenyl ring; and
X is either a carbonyl (CO) group or a sulfonyl (SO₂) group;
or a pharmaceutically acceptable salt thereof.

In another embodiment, the novel CMV inhibitor compounds of the present invention comprise a compound having the structure of Formula II:

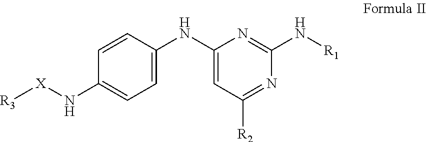

wherein:
R₁ is a substituted phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is a substituted phenyl or furan ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is either a carbonyl (CO) group, a sulfonyl (SO₂) group, or a NHCO group (part of a urea).

In another embodiment, the novel CMV inhibitor compounds of the present invention comprise a compound having the structure of Formula III:

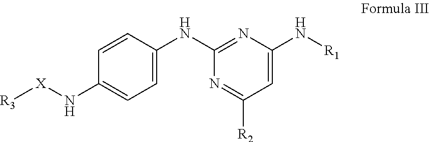

wherein:
R₁ is a phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is substituted phenyl ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is a carbonyl (CO) group.

Specific cytomegalovirus inhibitor compounds of the present invention are selected from:

| Cmpd # | Structure |
|---|---|
| MBXC- 4294 | 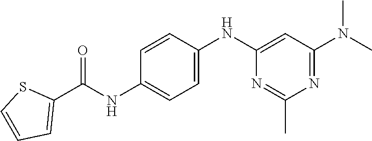 [SMILES: Cc1nc(Nc2ccc(NC(=O)c3cccs3)cc2)cc(N(C)C)n1] |
| MBXC- 4295 | 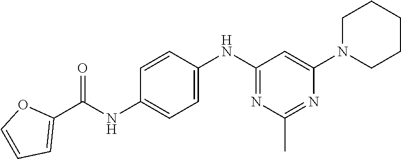 [SMILES: Cc1nc(Nc2ccc(NC(=O)c3ccco3)cc2)cc(N2CCCCC2)n1] |
| MBXC- 4297 | 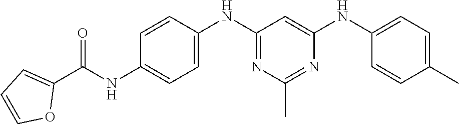 [SMILES: Cc1ccc(Nc2cc(Nc3ccc(NC(=O)c4ccco4)cc3)nc(C)n2)cc1] |
| MBXC- 4298 | 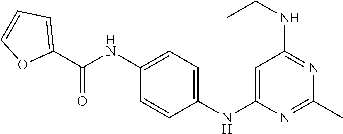 [SMILES: CCNc1cc(Nc2ccc(NC(=O)c3ccco3)cc2)nc(C)n1] |
| MBXC- 4299 | 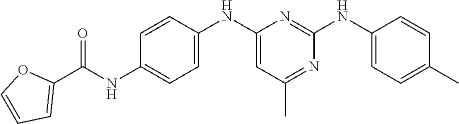 [SMILES: Cc1ccc(Nc2nc(C)cc(Nc3ccc(NC(=O)c4ccco4)cc3)n2)cc1] |
| MBXC- 4300 | 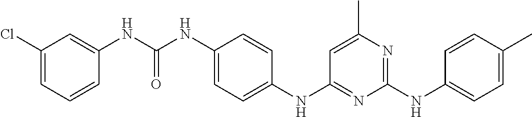 [SMILES: Cc1ccc(Nc2nc(C)cc(Nc3ccc(NC(=O)Nc4cccc(Cl)c4)cc3)n2)cc1] |
| MBXC- 4301 | 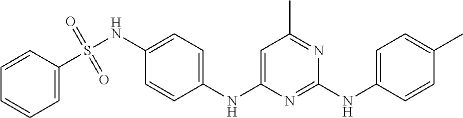 [SMILES: Cc1ccc(Nc2nc(C)cc(Nc3ccc(NS(=O)(=O)c4ccccc4)cc3)n2)cc1] |
| MBXC- 4302 | 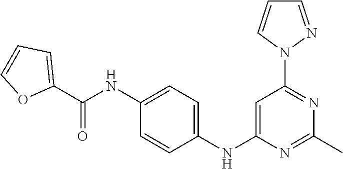 [SMILES: Cc1nc(Nc2ccc(NC(=O)c3ccco3)cc2)cc(-n2cccn2)n1] |
| MBXC- 4303 | 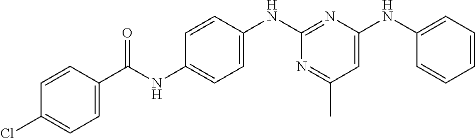 [SMILES: Cc1cc(Nc2ccccc2)nc(Nc2ccc(NC(=O)c3ccc(Cl)cc3)cc2)n1] |
| MBXC- 4304 | 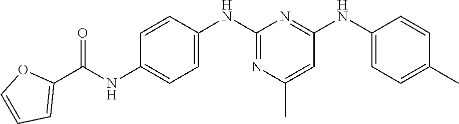 [SMILES: Cc1ccc(Nc2cc(C)nc(Nc3ccc(NC(=O)c4ccco4)cc3)n2)cc1] |
| MBXC- 4325 | 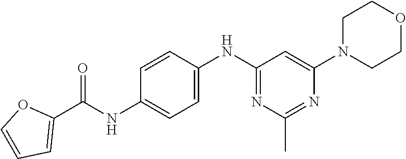 [SMILES: Cc1nc(Nc2ccc(NC(=O)c3ccco3)cc2)cc(N2CCOCC2)n1] |
| MBXC- 4330 | 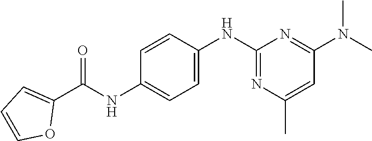 [SMILES: Cc1cc(N(C)C)nc(Nc2ccc(NC(=O)c3ccco3)cc2)n1] |
| MBXC- 4336 | 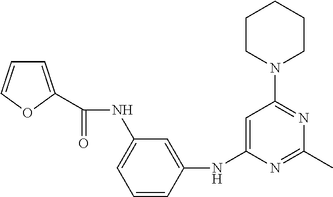 [SMILES: Cc1nc(Nc2cccc(NC(=O)c3ccco3)c2)cc(N2CCCCC2)n1] |
| MBX- 4991 | 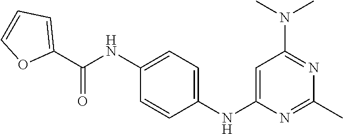 [SMILES: Cc1nc(Nc2ccc(NC(=O)c3ccco3)cc2)cc(N(C)C)n1] |
| MBX- 4992 | 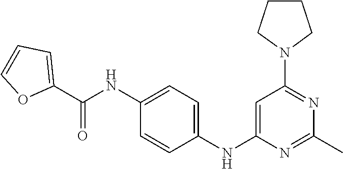 [SMILES: Cc1nc(Nc2ccc(NC(=O)c3ccco3)cc2)cc(N2CCCC2)n1] |

and pharmaceutically acceptable salts thereof.

Therefore, it is an object of the present invention to develop new anti-cytomegalovirus therapeutics to target the binding interaction between the virus and a host cell. Multiple routes of administration are envisioned for these drug-like molecules, and highly cost-effective production strategies can be easily achieved.

In one embodiment, the present invention is related to the discovery of novel organic small molecule inhibitors against cytomegalovirus entry into host cells. The inhibitors described herein are suitable for use in a composition for the treatment and/or prevention of cytomegalovirus infections in a mammal. More particularly, the inhibitors described herein are suitable for the treatment and/or prevention of cytomegalovirus infections in humans.

In another embodiment, the novel small molecule inhibitors described herein are suitable for use in a method for treating or preventing cytomegalovirus infections in a mammal by administration of the inhibitors of Formula I, Formula II, and/or Formula III described herein to a patient or subject in need thereof. In a preferred embodiment, the cytomegalovirus inhibitors described herein are suitable for use in a method for treating or preventing cytomegalovirus infections in humans.

The present invention is further directed to a method for the manufacture of a medicament for treating or preventing cytomegalovirus infection in mammals, in particular humans comprising combining one or more disclosed compounds of Formula I, Formula II, and/or Formula III of the present invention, products, or compositions with a pharmaceutically acceptable carrier or diluent. Thus, in one aspect, the invention relates to a method for manufacturing a medicament comprising combining at least one disclosed compound according to the present invention or at least one disclosed product with a pharmaceutically acceptable carrier or diluent.

In another embodiment, the present invention is directed to the use of the novel NAPA compounds described herein in a method for the manufacture of a medicament for treating or preventing CMV infection in mammals (e.g., humans) comprising combining one or more of the NAPA CMV inhibitor compounds of Formula I, Formula II, and/or Formula III of the present invention, products, or compositions with a pharmaceutically acceptable carrier or diluent.

In another embodiment, the present invention is directed to a compound for use in a method of treating or preventing a cytomegalovirus infection in a mammalian subject, the method comprising administering to a subject in need thereof an effective amount of a cytomegalovirus inhibitor compound having the structure of Formula I, Formula II, and/or Formula III.

In another embodiment, the present invention is directed to a composition comprising at least one compound for use in a method of inhibiting cytomegalovirus infection in a mammal, the method comprising administering an effective amount of a composition comprising at least one compound, the compound having the structure of Formula I, Formula II, and/or Formula III.

In another embodiment, the present invention is directed to a pharmaceutical composition comprising at least one cytomegalovirus inhibitor compound of Formula I, Formula II, and/or Formula III, and a pharmaceutically acceptable carrier or excipient.

In another embodiment, the present invention provides a method of treating, preventing, or reducing the likelihood that a transplant recipient, or prospective transplant recipient, will become infected with cytomegalovirus from a donor organ, tissue, or cell population, the method comprising contacting the organ or tissue in vitro or ex vivo with a Formula I, Formula II, and/or Formula III NAPA compound as described herein for a period of time and transplanting the treated organ or tissue into the transplant recipient. Transplant patients suitable for such treatment include, but are not limited to, liver transplant patients, kidney transplant patients, lung transplant patients, and bone marrow transplant patients.

Additional at-risk subjects suitable for treatment according to the method described herein include, but are not limited to, subjects at higher risk of CMV infection including HIV-positive individuals, patients with autoimmune disorders, neonates with extensive CNS disorders, patients with cardiovascular disease, cancer patients, and elderly patients.

In preferred embodiments, the novel NAPA compounds of the present invention exhibit potent antiviral activity against CMV strains (IC₅₀≤10 μM), favorable cytotoxicity, i.e., CC₅₀≥100 μM, optimal in vivo drug interaction, i.e., a minimal inhibition of CYP450 (<30% at 10 μM), favorable bioavailability, i.e., a Caco-2 permeability value (Pₐₚₚ) of >1×10⁻⁶ Cm/sec, and a selectivity index (CC₅₀/IC₅₀)≥100.

To identify inhibitors that prevent entry of the cytomegalovirus into host cells, as described herein, the assay was optimized for rapid screening of a large (>112,000) library of structurally diverse small molecules to identify potent inhibitors (IC₅₀<10 μM) of cytomegalovirus and having a minimal mammalian cytotoxicity (CC₅₀) of preferably ≥100 μM.

Unless otherwise indicated, it is understood that description of the use of a cytomegalovirus inhibitor compound in a composition or method also encompasses the embodiment wherein one or a combination of two or more cytomegalovirus NAPA inhibitor compounds described herein are employed as the source of cytomegalovirus inhibitory activity in the composition or method of the invention.

### Compositions and Methods

The compositions and methods of the presently disclosed invention are useful for treating and/or preventing cytomegalovirus infections in that they inhibit the onset, growth, or spread of the condition, cause regression of the condition, cure the condition, or otherwise improve the general well-being of a mammalian subject, preferably a human, afflicted with, or at risk of, contracting a cytomegalovirus infection. Thus, in accordance with the presently disclosed subject matter, the terms ‘treat’, ‘treating’, and grammatical variations thereof, as well as the phrase ‘method of treating’, and ‘use for treating’ are meant to encompass any desired therapeutic intervention, including but not limited to a method for treating an existing cytomegalovirus infection in a subject, and a method for the prophylaxis (i.e., prevention) of cytomegalovirus infection, such as in a subject that has been exposed to the virus as disclosed herein or that has an expectation of being exposed to the virus as disclosed herein.

Pharmaceutical compositions according to the invention comprise a NAPA cytomegalovirus inhibitor compound of Formula I, Formula II, and/or Formula III as described herein, or a pharmaceutically acceptable salt thereof, as the ‘active ingredient’ and a pharmaceutically acceptable carrier (or ‘vehicle’), which may be a liquid, solid, or semi-solid compound.

In some embodiments, the presently disclosed subject matter is related to a method of treating or preventing a cytomegalovirus infection in a subject in need of treatment thereof wherein the method comprises administering to the subject an effective amount of a composition comprising a compound of Formula I, Formula II, and/or Formula III. The compound or compounds may be administered alone or optionally in combination with one or more additional antiviral agents.

Preferably, the cytomegalovirus inhibitor compounds described herein can be administered as pharmaceutically acceptable salts. Such pharmaceutically acceptable salts include the gluconate, lactate, acetate, tartarate, citrate, phosphate, maleate, borate, nitrate, sulfate, and hydrochloride salts. The salts of the compounds described herein can be prepared, for example, by reacting the base compound with the desired acid in solution. After the reaction is complete, the salts are crystallized from solution by the addition of an appropriate amount of solvent in which the salt is insoluble. In some embodiments, the hydrochloride salt is made by passing hydrogen chloride gas into an ethanolic solution of the free base. Accordingly, in some embodiments, the pharmaceutically acceptable salt is a hydrochloride salt.

In another embodiment, the compounds are formulated into a pharmaceutically acceptable carrier or excipient for administration to a subject in need thereof. In another embodiment, the compounds may be formulated into a pharmaceutical formulation and further comprise an additional antiviral compound. In another embodiment, the pharmaceutical formulation may be formulated to be administered orally, parenterally, or topically.

It is preferable to develop an orally active therapeutic, since that is the most convenient and rapid method to administer a drug to a large, exposed population in case of pandemic. However, it is also expected that the cytomegalovirus inhibitors described herein will be suitable for intravenous (i.v.) administration, because it is envisioned that in case of a natural outbreak the infected patients may require i.v. administration. Therefore, the inhibitors described herein will provide an effective, safe, and easy therapeutic option for any newly emerged pandemic strain(s).

In another aspect, the invention relates to pharmaceutical compositions comprising an effective amount of one or more compounds according to Formula I, Formula II, and/or Formula III herein, or a pharmaceutically acceptable salt, solvate, hydrate, or polymorph thereof, and a pharmaceutically acceptable carrier. That is, a pharmaceutical composition can be provided comprising at least one disclosed compound of the present invention, at least one product of a disclosed method, or a pharmaceutically acceptable salt, solvate, hydrate, or polymorph thereof, and a pharmaceutically acceptable carrier. In a further aspect, the effective amount is a therapeutically effective amount. In a still further aspect, the effective amount is a prophylactically effective amount.

In a further aspect, the pharmaceutical composition is a solid dosage form selected from a capsule, a tablet, a pill, a powder, a granule, an effervescing granule, a gel, a paste, a troche, and a pastille. In a still further aspect, the pharmaceutical composition is a liquid dosage form selected from an emulsion, a solution, a suspension, a syrup, and an elixir.

In another embodiment, the pharmaceutical composition of the present invention comprises a pharmaceutically acceptable carrier; an effective amount of at least one disclosed compound of the present invention; or a pharmaceutically acceptable salt, solvate, or polymorph thereof; and further comprises a second active agent. In a further aspect, the second active agent is an antiviral agent.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, palmoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion.

In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In another aspect, the invention relates to a kit comprising at least one compound according to Formula I, Formula II, and/or Formula III herein, or a pharmaceutically acceptable salt, solvate, or polymorph thereof; and one or more of:
a) at least one agent known to inhibit cytomegalovirus;
b) optionally at least one additional agent known to have antiviral activity;
c) instructions for treating a cytomegalovirus related disease;
d) instructions for administering the compound in connection with treating a cytomegalovirus infection; or
e) instructions for administering the compound with at least one agent known to treat a cytomegalovirus related disease.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound of the present invention and/or product and another component for delivery to a patient.

In a further aspect, the kit further comprises a plurality of dosage forms, the plurality comprising one or more doses; wherein each dose comprises an amount of the compound and the agent known to have antiviral activity. In another aspect, the kit further comprises a plurality of dosage forms, the plurality comprising one or more doses; wherein each dose comprises an effective amount of the compound and the agent known to have antiviral activity.

### Examples

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Cell Lines and Viruses:

MRC5 and NHDF (ATCC (?catalog #?, Manassas, VA) cells were cultured in DMEM (Corning #10-013-CV) with 10% FBS, 1 mM HEPES (Corning, #25-060-CI), 100U/mL penicillin and 100 g/mL streptomycin (Corning, #30-002-CI).

ARPE-19 human retinal epithelial cells (ATCC #CRL-2302) were cultured in DMEM/F-12 medium (Gibco, #11765-054) at 1:1 ratio with FBS, HEPES and Pen/Strep.

HRT-8/SVneo cells, human trophoblasts, were cultured in RPMI with FBS, HEPES and Pen/Strep.

HCMV strain AD169 and a repaired AD169 (denoted BADrUL131-C4) containing the UL131-UL128 open reading frame of the HCMV strain TR and expressing the reporter EGFP (AD169^{R}) (40) were propagated as described (Gardner et al. supra, 2015).

HCMV strains Merlin, Towne, TR, and TB40/E were propagated in the same manner. Infectious virus yield was assayed on fibroblasts by median tissue culture infective dose (TCID50).

Compounds: All compounds were obtained from ChemDiv (San Diego, CA) and used as received.

| Compound # | ChemDiv catalog # |
|---|---|
| 4294 | D430-0316 |
| 4295 | D430-0426 |
| 4297 | D430-0522 |
| 4298 | D576-0052 |
| 4299 | D576-0288 |
| 4300 | D577-0239 |
| 4301 | D578-0138 |
| 4302 | F5564-0132 |
| 4303 | G869-0316 |
| 4304 | G869-0246 |
| 4325 | D430-0478 |
| 4330 | D430-0320 |
| 4336 | D430-0426 |
| 4991 | G869-0411 |
| 4992 | D430-0374 |

### Example 1. Materials and Methods

Fibroblasts and ARPE-19 cells (10⁴) were plated in 96-well plates (Greiner, Monroe, NC). The following day the cells were pretreated for 1 hour at 37° C. with increasing concentrations of the respective novel NAPA compounds described herein or DMSO in triplicate prior to infection with the respective viruses. At 18 hours post infection (hpi) cells were stained using a rabbit anti-IE1 antibody followed by an anti-rabbit Alexa 647 and measured using a Celigo Cytometer (Parsons et al., Antiviral Res., 193:105124 (2021); Stein et al., Nat. Commun., 10:2699 (2019)). Using the DMSO treated cells as 100% infection, the percent infection of the NAPA compound-treated cells was determined. The half maximal inhibitory (IC50) values were calculated using Prism9's nonlinear fit [inhibitor]-vs-response (four parameters) analysis of these averages and extrapolation to the concentration that would produce 50% infection relative to DMSO treatment.

### Western Blotting Analysis.

Total cell lysates from virus infected NHDF cells treated with compounds were created through SDS-lysis (10⁴/100 uL of 1% SDS) of at least three rounds of heating at 95° C. for 3 min and vigorous agitation. The total cell lysates were resolved using an SDS-polyacrylamide gel (12.5%), transferred to a PVDF membrane using a semi-dry apparatus. The membrane was then incubated with 1% BSA/PBS for 1 hr at room temperature followed by incubation with anti-IE1 and anti-GAPDH. Finally, the respective secondary antibody conjugated with HRP was incubated with membrane followed by detection using ECL reagents (Millipore).

### Time-of-Addition Experiments.

NHDF cells (10⁴) were plated in a 96-well plate overnight. A working stock of MBX-4992 at 20 μM was used as a 2×to achieve a final concentration of 10 μM. MBX-4992 was added to wells in quintuplicate at the designated time points relative to virus infection (−60 to 90 minutes post infection) with AD169 at an MOI of 0.2. To control for volume change during the infection, DMSO-containing media was utilized. At 18 hpi, the cells were fixed with 4% paraformaldehyde, stained with a rabbit anti-IE1 antibody followed by an anti-rabbit Alexa 647, and analyzed using the Celigo cytometer. Using the −60 DMSO treated cells as 100% infectivity, the percent relative to maximum infection was determined from cells treated with drug at different time points.

### Pre-Incubation Studies.

### Virus-Pre-Incubation

NHDF cells (10⁴) plated overnight in a 96-well plate were incubated at 4° C. for 30 minutes with AD169^{R} CMV virus pretreated with MBX-4992 (10 μM), Heparin (50 μg/mL) or DMSO for 1 hour at room temperature (in triplicate). For 2/3^{rd} of the samples, media was aspirated and half of the wells received DMSO media, regardless of which drug the virus was exposed to, or MBX-4992 (10 μM), or Heparin (50 μg/mL) overnight. At 18 hpi, the cells were fixed with 4% paraformaldehyde, stained with a rabbit anti-IE1 antibody followed by an anti-rabbit Alexa 647, and analyzed using the Celigo cytometer. The DMSO condition that was not aspirated post binding was set as 100% infection.

### Cells-Pre-Incubation

NHDF cells (10⁴) plated overnight in a 96-well plate were pretreated with MBX-4992 (10 μM), heparin (50 μg/mL) or DMSO for 1 hour at 37° C. The plate was then placed at 4° C. for 5 minutes followed by the addition of AD169^{R} CMV virus for 30 minutes. 2/3^{rd} of the samples were aspirated post binding. Half of those samples received DMSO media, regardless of the drug the cells were previously exposed to. The remaining wells received either MBX-4992 (10 μM) or Heparin (50 μg/mL) overnight. At 18 hpi, the cells were fixed with 4% paraformaldehyde, stained with a rabbit anti-IE1 antibody followed by an anti-rabbit Alexa 647, and analyzed using the Celigo cytometer. The DMSO condition that was not aspirated post binding was set as 100% infection.

### Cytotoxicity Analysis:

The CellTiter Glo Luminescent Assay (Promega Inc, Madison, WI) was performed according to the manufacturer's instruction. NHDF cells (10⁴) were plated in a 96-well plate overnight and treated with MBX-4992 or MXC-4336 (0-50 M) in triplicate for up to 6 days. Cyclohexamide (50 μg/mL) was used as a control. CellTiter Glo substrate was added in a 1:1 ratio with media and CellTiter-Glo reagent followed by the analysis of luciferase activity (relative light units, RLU) using a BioTek Synergy H1 microplate reader. In parallel, an identical plate was labelled with Hoescht reagent (25 μg/mL) to quantify the cell number using a Celigo Cytometer.

### Dissemination Analysis:

For the prophylactic treatment analysis, the plaque assay was performed on ARPE-19 and NHDF cells (4×10⁴) in a 24-well plate. The cells were pretreated for 1 hour with either MBX-4992 or MBXC-4336 (1-50 μM), DMSO, or ganciclovir (2.5 μM) and followed by virus infection with AD169^{R}. Following a 2-hour incubation at 37° C., the inoculum was removed and cells were overlaid with 1% low melt sea agarose. Upon solidification of the agarose, MBX-4992, MBXC-4336, DMSO, or ganciclovir in media was added at the indicated concentration. The drugs were replaced every 3 days. The cells were examined using Brightfield and GFP fluorescence to quantify the number and size of virus plaques using a Celigo Cytometer at 10 dpi.

For therapeutic treatment, ARPE-19 cells (4×10⁴) were plated in a 24-well plate. 9 of the 24 wells were pretreated with either MBX-4992 or MBXC-4336 (10 μM), DMSO or GCV (2.5 M) for 1 hour and then infected with AD169^{R} virus for a 2-hour incubation at 37° C. The inoculum was then removed and cells were overlaid with 1% low melt sea agarose. At 48 hours post infection, MBX-4992 (1-50 μM), or MBXC-4336 (1-50 μM) were added to the cells and was replaced with the respective drugs every 3 days. As controls, MBX-4992 or MBXC-4336 (10 μM) were used to pre-treat (PT) the cells with compound. The cells were examined using brightfield and YFP fluorescence to quantify the number and plaques using the Celigo Cytometer at 14. The viral clusters were referred to as plaques based on average cluster area (5,000 μm² or 10,000 μm²).

### Example 2. High-Content Screen (HCS) Using AD169^{IE2-YFP}-Infected Fibroblasts to Identify Hit Compounds

The CMV IE1 and IE2 gene products are translated within 3-6 hours post infection (hpi) and function to stimulate viral promoters to initiate transcription of early and late viral genes (Cherrington et al., J. Virol., 63:1435-40 (1989); Meier et al., J. Virol., 71:1246-1255 (1997)). The CMV AD169 variant that expresses a viral protein chimera consisting of IE2-YFP (AD1691E2/YFP) was utilized to quantify virus infection in a high-throughput screen by a robust fluorescent signal localized exclusively to the nucleus (8-24 hpi) (Gardner et al., supra (2015); Cohen et al., Viruses, 8 (10): 295 (2016); Cohen et al., J. Virol., 90:10715-10727 (2016)). Using the AD1691E2-YFP reporter virus assay, a collection of the compound libraries (>112,000 compounds) was screened for inhibitors of the early stages of CMV replication using a confocal fluorescence microscopic plate reader (BioTek Cytation3). Infection was calculated as a percentage of nuclei containing YFP and cycloheximide (8 μM) was used as a positive control. The Z′ factor for the optimized assay was >0.5 which was sufficient to initiate screening.

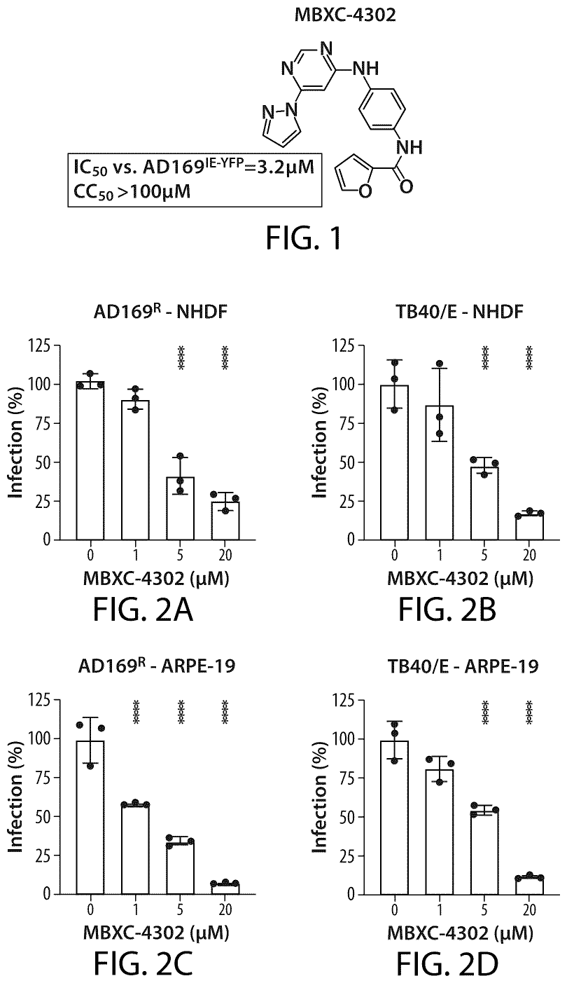

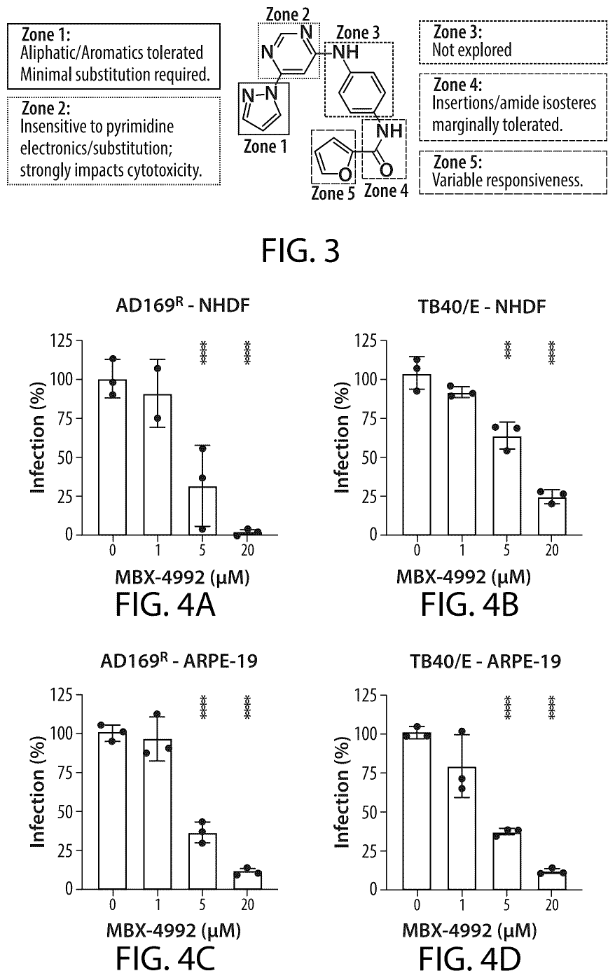

We evaluated 300 primary hits using an assay funnel designed to prioritize compounds based on antiviral activity, specificity, and drug-like properties. This identified two candidates for hit-to-lead optimization that were not Pan Assay Interference compounds (PAINS) (Baell et al., J. Med. Chem., 53:2719-40 (2010); Capuzzi et al., J. Chem. Inf. Model 57:417-427 (2017); Dahlin et al., Assay Drug Dev. Technol., 14:168-174 (2016)). The primary hit-to-lead series is exemplified by MBXC-4302 (see FIG. 1), an N-arylpyrimidinamine (NAPA) which exhibits potent anti-CMV activity (IC₅₀=3.2 μM), limited cytotoxicity (CC₅₀>100 μM), favorable in vitro ADME properties, confirmed lifecycle specificity, and well-defined preliminary structure activity relationships (FIG. 3 and Table 1). Table 1 shows the potency and in vitro ADME values of select NAPA analogs of the present invention. The NAPA series was chosen as the primary hit-to-lead series due to its synthetic accessibility and the >2-log range in potency observed in the preliminary SAR that provides a clear pathway to improving antiviral activity and drug-like properties.

TABLE 1
Activity and cytotoxicity of NAPA compounds with varying substituents.

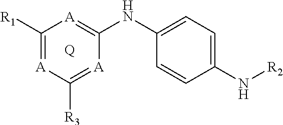

Q =

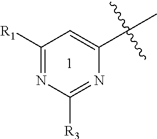

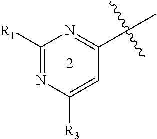

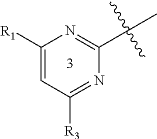

| | # | CMPD# | R₁ | | R₂ | Q(R₃) | IC₅₀ª | CC₅₀^{b} | Sol^{c} | MLMS^{d} | CYP^{e} |
|---|---|---|---|---|---|---|---|---|---|---|---|
| Zone 1 | 1 | MBXC-4298 | | 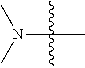 [SMILES: *N(C)C] | | 2(Me) | 3.7 | 29 | 200 | 64 | 50 |
| | 2 | MBX-4991 | 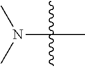 [SMILES: *N(C)C] | | | 2(Me) | 2.3 | >100 | >200 | 65 | 18 |
| | 3 | MBX-4992 | | 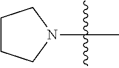 [SMILES: *N1CCCC1] | | 1(Me) | 1.72 | >100 | >200 | 49 | <10 |
| | 4 | MBXC-4295 | 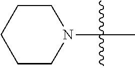 [SMILES: *N1CCCCC1] | | | 1(Me) | 0.46 | 54 | 50 | 36 | <10 |
| | 5 | MBXC-4302 | | 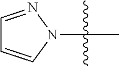 [SMILES: *n1cccn1] | 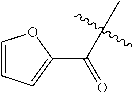 [SMILES: *C(=O)c1ccco1] | 1(H) | 3.2 | >100 | 12.5 | 62 | <10 |
| Zone 2 | 6 | MBXC-4297 | | | | 1(Me) | 0.37 | 21 | 25 | 44 | <10 |

| | | | | | | | | |
|---|---|---|---|---|---|---|---|---|
| 7 8 | MBXC-4299 MBXC-4304 | 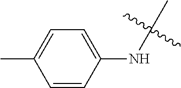 [SMILES: *Nc1ccc(C)cc1] | 2(Me) 3(Me) | 0.32 0.48 | 5.5 5.8 | 12.5 12.5 | 68 63 | <10 16 |

| | | | | | | | | | |
|---|---|---|---|---|---|---|---|---|---|
| Zone 4 | 9 10 | MBXC-4301 MBXC-4300 | 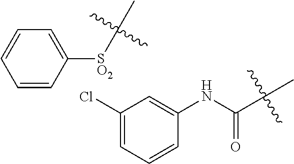 | 2(Me) 2(Me) | 24 3.8 | 11 2.7 | N/A 3.1 | N/A 60 | N/A 10 |

| | | | | | | | | | | |
|---|---|---|---|---|---|---|---|---|---|---|
| Zone 5 | 11 | MBXC-4303 | 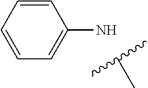 | 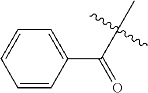 [SMILES: *C(=O)c1ccccc1] | 3(Me) | 20 | 7 | N/A | N/A | N/A |
| | 12 | MBXC-4294 | 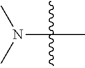 [SMILES: *N(C)C] | 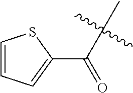 [SMILES: *C(=O)c1cccs1] | 1(Me) | >40 | 57 | N/A | N/A | N/A |

^{a}All values in μM.
^{b}Against Hela cells for 3 d.
^{c}Maximum solubility in water determined by nephlometry.
^{d}Murine liver microsom stability, % remaining after 30 min at 37° C. in the presence of NADPH.
^{e}% inhibition of CYP3A4 at 10 μM.
^{f}Scatterplot of IC₅₀ vs. CC₅₀ for all analogs examined.

### Example 3. MBXC-4302 Broadly Limits Virus Infection in Diverse Cell Types

To further evaluate the inhibitory properties of MBXC-4302 against CMV, we utilized the AD169 CMV virus strain containing the UL131-UL128 open reading frame of the HCMV strain TR and expressing EGFP (AD169^{R}) (Wang D. and Shenk T., Proc. Nat. Acad. Sci., USA, 102:18153-18158 (2005)) and TB40/E infection of human fibroblasts (NHDF) and ARPE-19 epithelial cells in the presence of increasing concentration of compound (FIG. 2A-D). Infection was analyzed 24 hours post infection (hpi) by measuring GFP fluorescence encoded by AD169^{R} or using an anti-IE1 antibody for staining TB40/E infected cells.

MBXC-4302 inhibited AD169^{R}-infection of NHDF and ARPE-19 cells with IC50 values of 3 μM and 5.8 μM, respectively (FIGS. 2A and C). In addition, the MBXC-4302 IC50 values of TB40/E-infected NHDF and ARPE-19 cells was ˜5.3 μM, respectively (FIGS. 2B and D). Taken together, the data demonstrate that MBXC-4302 is a potent inhibitor of CMV infection.

### Example 4. Inhibitory Analysis of NAPA Analogs

We next examined >30 MBXC-4302 analogs, establishing a responsive structure activity relationship (SAR) and improving potency 10-fold (overall trends are highlighted in FIG. 3). The selectivity index (SI) varies widely, ranging from 12 to >100, consistent with an antiviral mechanism that is independent from any cytotoxic mechanisms. A representative CMV inhibitory compound, MBX-4992, exhibits a low μM potency, a >50-fold SI, and high solubility. Additional preliminary studies with other NAPA analogs demonstrated a responsive SAR and identified potential strategies for further chemical optimization of potency, selectivity and ADME properties (data not shown). Advantageously, NAPA analogs can be readily synthesized via a straightforward synthetic route.

### Example 5. NAPA Analog MBX-4992 Broadly Inhibits a CMV Infection

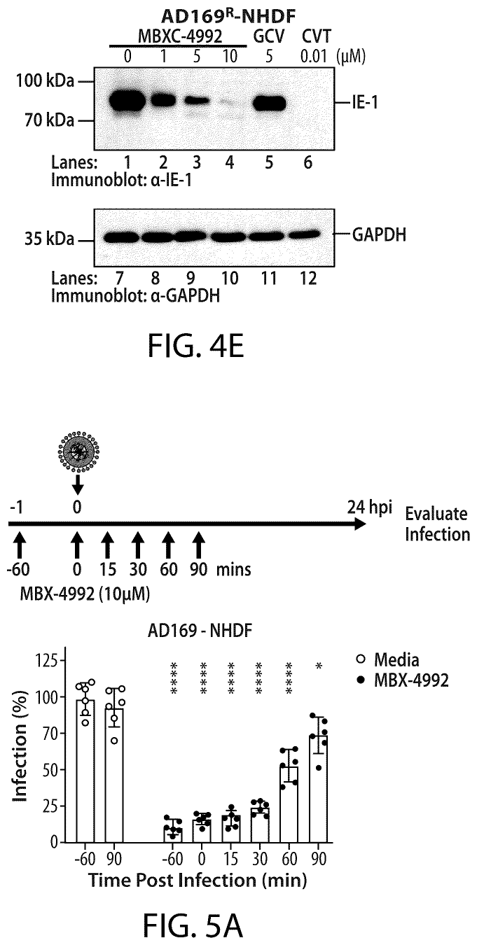

We next evaluated compound MBX-4992 for inhibition against CMV (FIG. 4). NHDF and ARPE-19 cells were pre-incubated with DMSO or increasing concentrations of MBX-4992 (1, 5, and 20 μM) then infected with CMV strains AD169^{R} and TB40/E and analyzed for infection at 24 hpi by immunostaining for IE1. Results showed a concentration dependent decrease in virus infection with IC50 values ranging from 1.9-4.8 μM (FIG. 4A-D).

MBX-4992 was similarly effective at limiting IE1 expression when analyzed 2 days post-infection of AD169- and AD169^{R}-infected fibroblasts (FIG. 10A &B). MBX-4992 limited infection of CMV strains AD169, TB40/E^{Flag-YFP}, Towne, and TR in fibroblasts with IC50 values ranging from 0.5-8 μM, respectively. (FIG. 11A-G). Also, MBX-4992 limited infection of TB40/E and AD169^{R} infection of HRT8svNEO trophoblasts (FIG. 11I-J). The IC50 values were similar in diverse cell types and among the different strains further supporting that NAPA compounds are effective at inhibiting CMV infection.

Next, inhibition of the expression of IE1 in AD169^{R}-infected cells treated with MBX-4992 was examined by immunoblot analysis (FIG. 4E). A concentration dependent decrease in IE1 expression was observed upon treatment with MBX-4992 (FIG. 4E, lanes 1-4). As expected, treatment with ganciclovir (GCV) did not impact infection (FIG. 4E, lane 5), while convallatoxin (CVT) treatment prevented infection (Cohen et al., J. Virol., 90:10715-10727 (2016)) (FIG. 4E, lane 6). The anti-GAPDH immunoblot demonstrated equivalent protein loading (FIG. 4E, lanes 7-12). A similar immunoblot result was observed in virus infected-APRE-19 cells (FIG. 10C). Collectively, these findings further indicate that MBX-4992 effectively prevents the expression of the immediate-early genes during a CMV infection.

To determine the specificity of MBX-4992, additional neutralization experiments were conducted using a panel of herpes viruses performed in the presence of MBX-4992. Table 2 shows the spectrum of antiviral activity of MBX-4992 against herpes viruses. As seen in Table 2, MBX-4992 exhibited potent antiviral activity against human CMV (IC₅₀ 1.7 μM) that is comparable to that of GCV (IC₅₀ 0.9 μM). In addition, MBX-4992 was effective against a GCV-resistant strain of human CMV (IC₅₀ 1.8 μM). Importantly, MBX-4992 demonstrated efficacy against mouse CMV (IC₅₀ 4.5 μM) indicating it has specificity against cytomegaloviruses. Collectively, the antiviral activity of MBX-4992 is equivalent to that of GCV indicating specific inhibition of CMV replication.

TABLE 2
Activity of MBX-4992 against various herpes viruses.

| | | MBX-4992 | | | GCV |
|---|---|---|---|---|---|
| Virus | Strain | IC₅₀* | CC₅₀* | SI | IC₅₀* |
| HSV-1 | E-377 | >30 | 60.6 | <2 | ND |
| EBV | Akata | 30.9 | >100 | >3 | ND |
| HHV-6B | Z29 | 43.7 | >100 | >2 | ND |
| HHV-8 | BCBL-1 | 10 | >100 | >10 | ND |
| HSV-2 | G | >150 | >150 | 1 | ND |
| HCMV | AD169 | 1.7 | >150 | >88 | 0.9 |
| HCMV | GDGr K17 (GCV^{R}) | 1.8 | >151 | >83 | 10.4 |
| MCMV | Smith | 4.5 | >150 | >33 | 0.2 |
| GpCMV | 22122 | >30 | 118.8 | <4 | ND |
| VZV | Ellen | >30 | 139.2 | <5 | ND |

*concentrations are in μM
Abbreviations:
HSV-1. Herpes simplex virus 1;
EBV. Epstein-Barrvirus;
HHV-6. Human herpes virus 6B;
HHV-8. Human herpes virus 8;
HSV-2. Herpes simplex virus 2;
HCMV. Human cytomegalovirus;
MCMV. Murine cytomegalovirus;
GpCMV. Guinea pig cytomegalovirus;
VZV. Varicella-Zoster virus

### Example 6. NAPA Compound Targets an Early Step of Infection

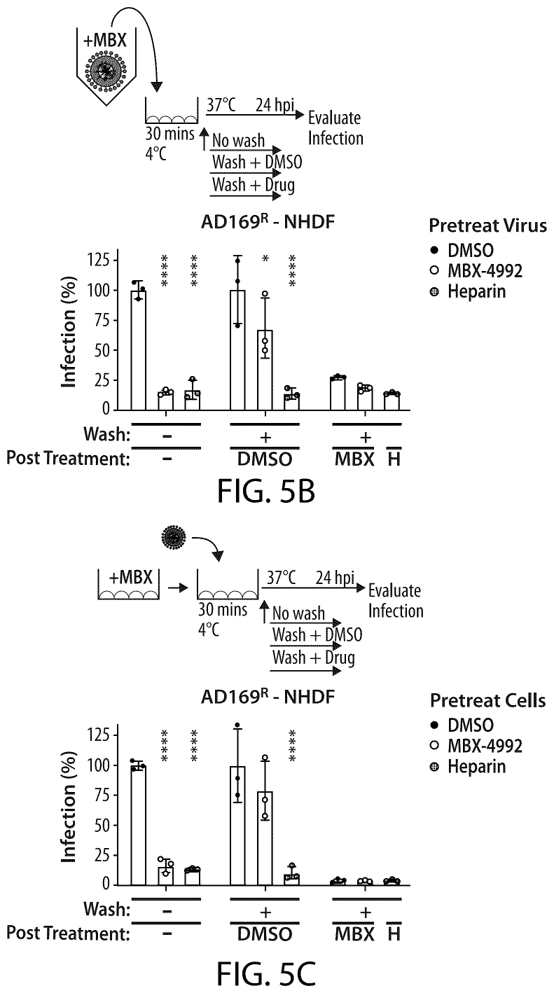

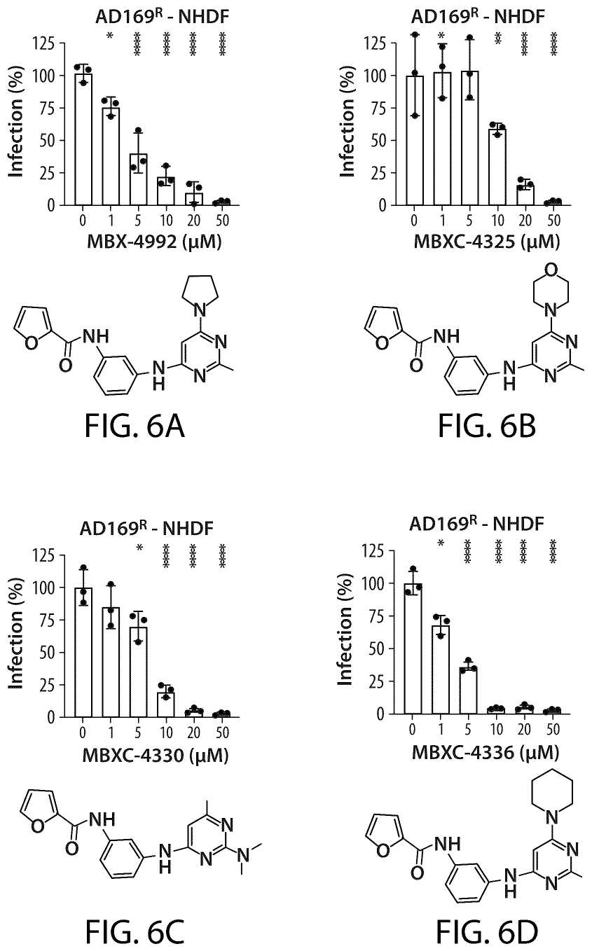

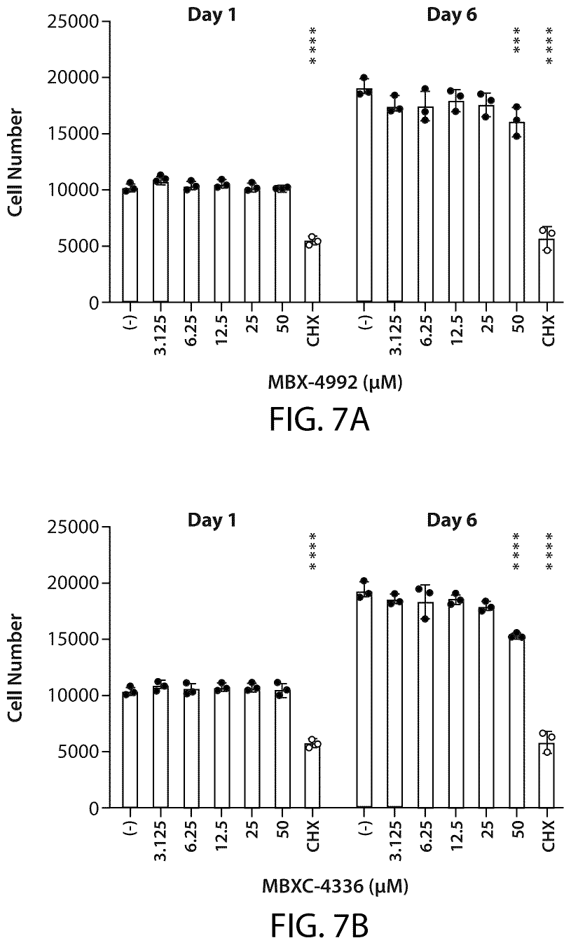

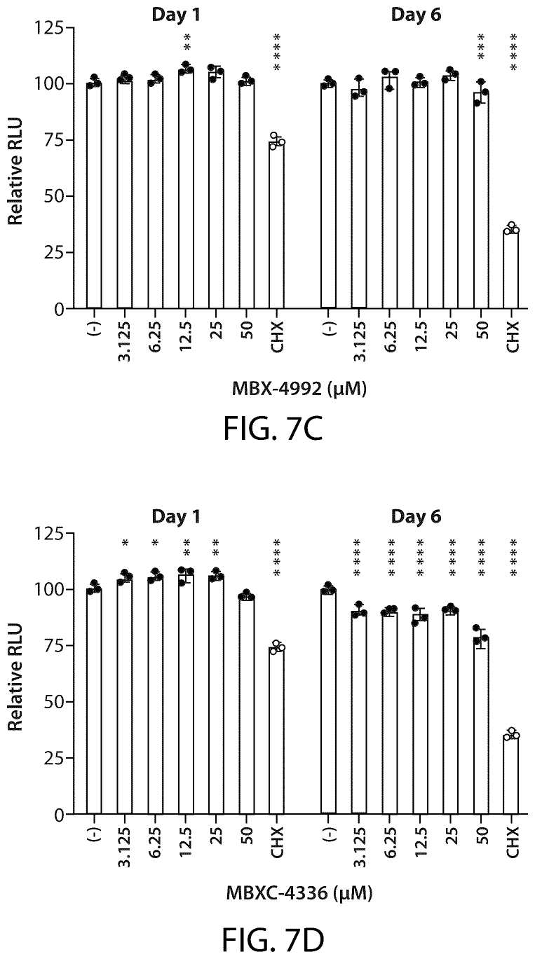

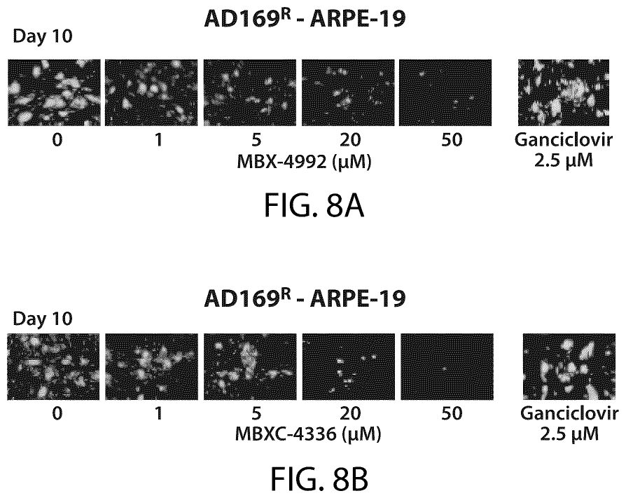

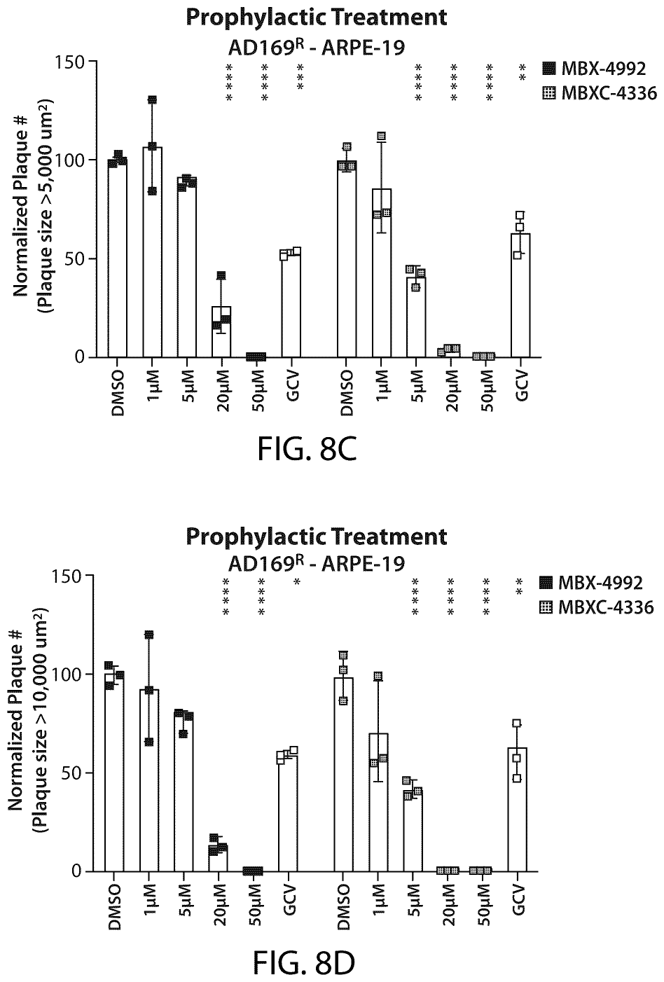

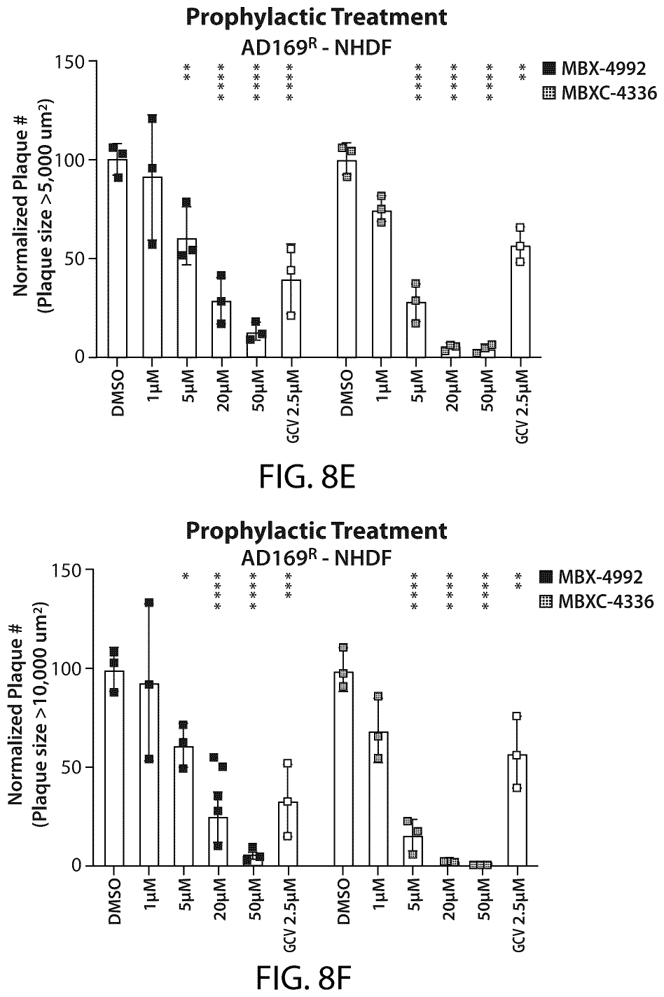

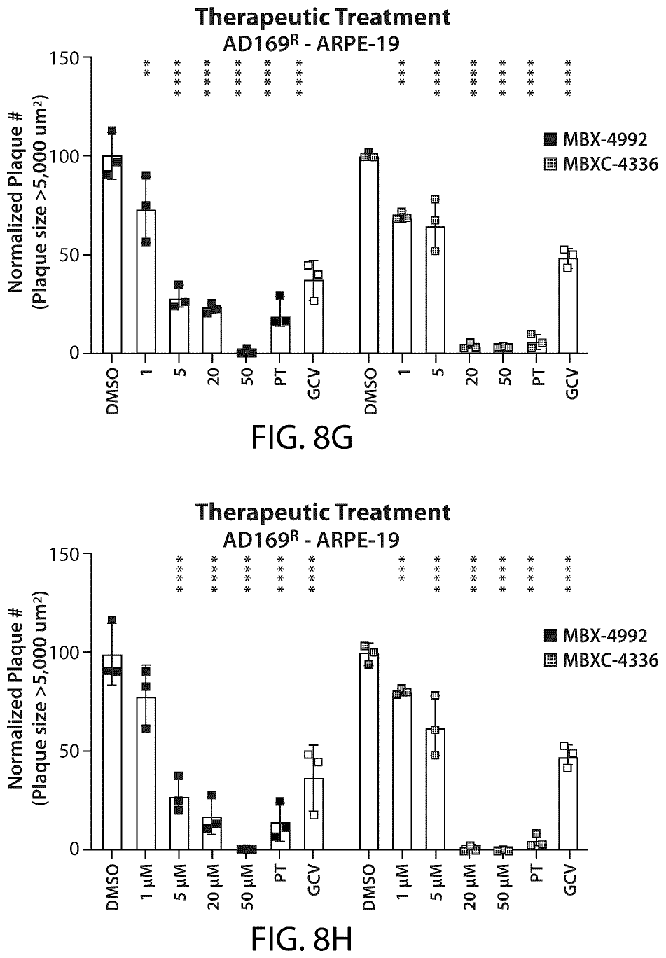

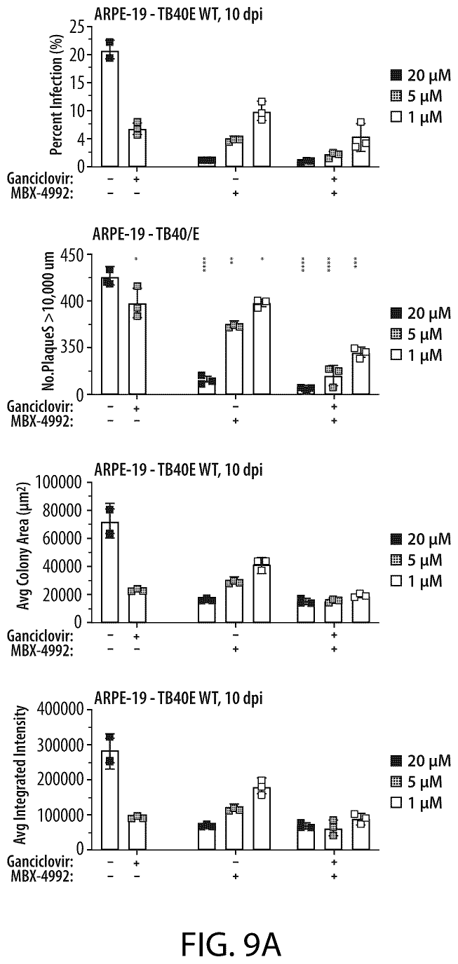

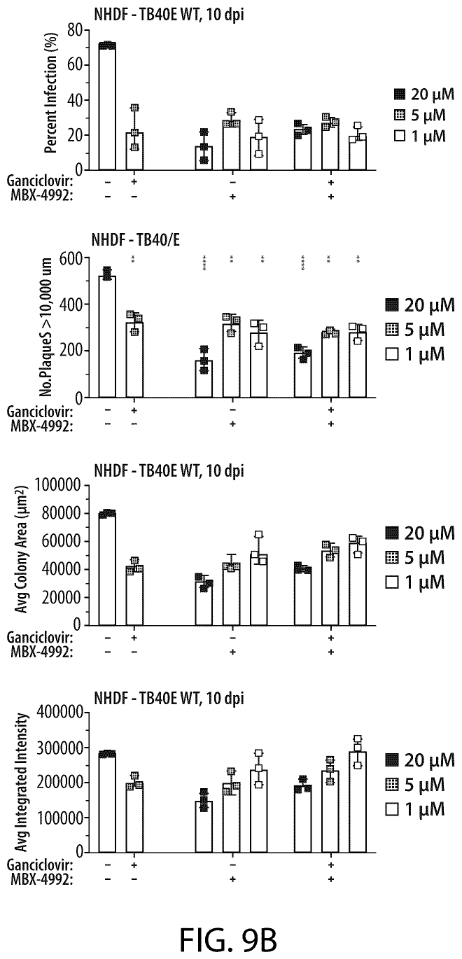

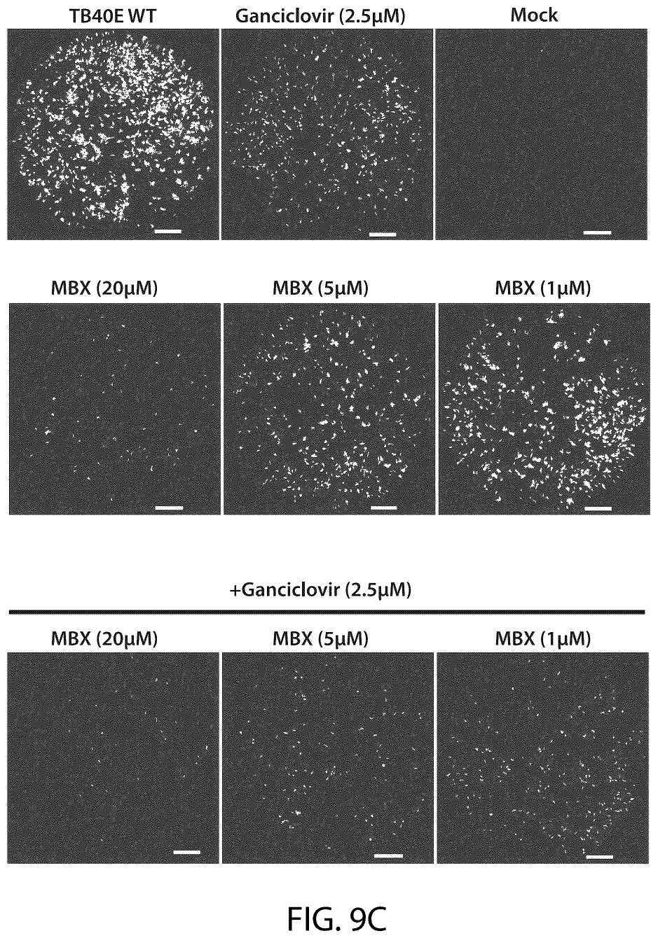

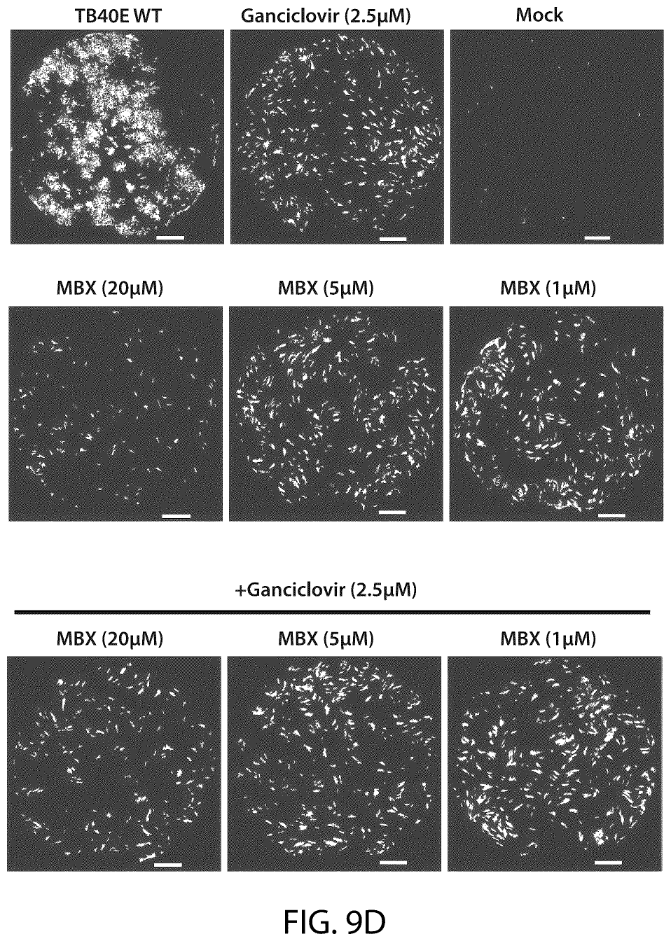

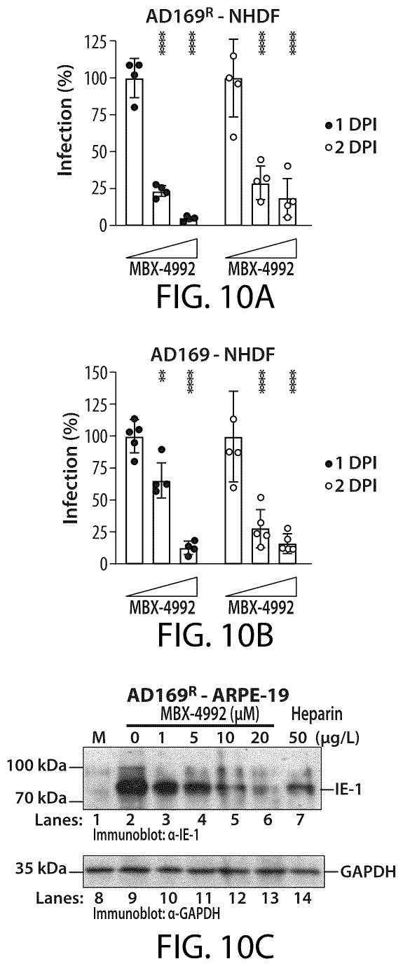

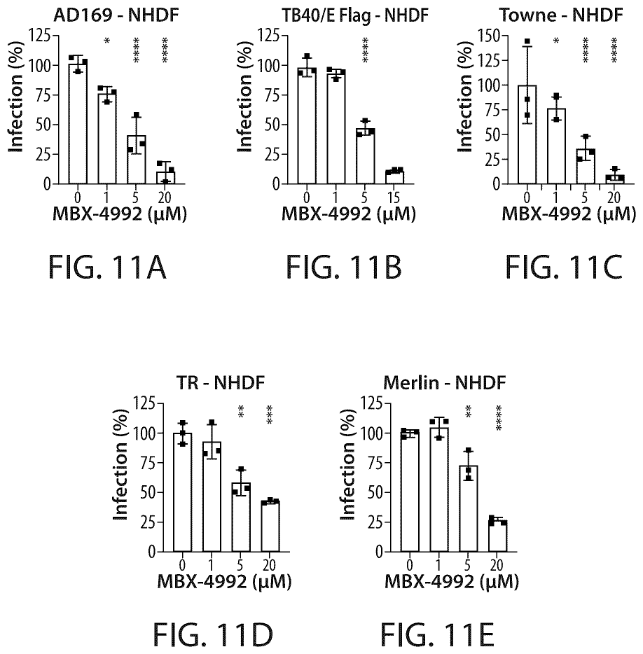

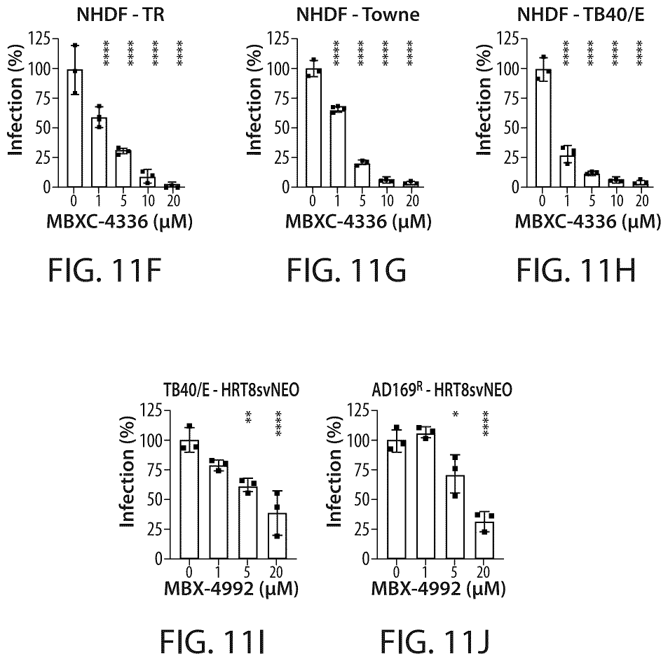

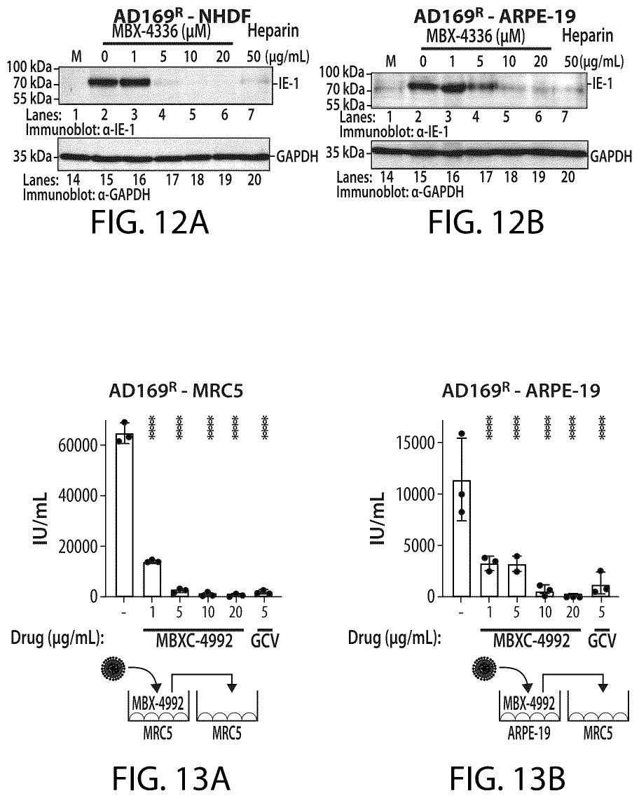

To further analyze the step of CMV entry targeted by NAPA compound MBX-4992, a time of addition assay was performed. The results are shown in FIG. 5A. MBX-4992 (10 μM) was added at various times during MRC5 infection (−60, 0, 15, 30, 60, and 90 minutes post-infection (mpi)) with AD169 CMV virus and viral infection was quantified at 24 hpi. As a control, DMSO was added at −60 and 90 mpi.

Results show that MBX-4992 was most effective at preventing infection when introduced prior to and within 30 mpi. The inhibitory effect of MBX-4992 started to diminish at 60 mpi and further at 90 hpi. Given that CMV binding and fusion occurs for up to 2 hours after virus addition, MBX-4992 likely advantageously inhibits the binding and/or the fusion steps of virus entry based on the kinetics of inhibition.

To further characterize the mechanism of action, pre-incubation/washout studies were performed using AD169^{R} and MBX-4992. The results are shown in FIG. 5B. Virus was pre-incubated with DMSO, MBX-4992 (10 μM), or heparin (50 μg/ml) for 30 mins then added to fibroblasts at 4° C. and incubated for 30 min to allow for virus to bind. The low temperature of incubation allows for binding but not fusion with the plasma membrane and entry of the virions. The virus-containing media was then either washed out or left unchanged as indicated and replaced with media containing DMSO, MBX-4992, or heparin followed by incubation at 37° C., allowing for virus entry. At 18 hpi, cells were immunostained with an anti-IE1 antibody as a readout for infection and quantified using a Celigo cytometer. The percent infection was normalized to DMSO-treated virus with no wash step.

As expected, pre-treatment/no wash-out and pre-treatment/treatment with MBX-4992 or heparin decreased infection by >90%. Interestingly, washing out MBX-4992 decreased virus infection by only ˜25% suggesting MBX-4992 has a minor impact on virus binding. The heparin pre-treatment and wash-out provided a positive control for inhibition. Together, these in vitro experiments suggest that MBX-4992 mainly influences virus infection in a post-binding step.

We next examined whether pre-treating cells with MBX-4992 can limit virus infection. The results are shown in FIG. 5C. Cells were pre-treated with MBX-4992 or heparin for 30 mins followed by the addition of AD169^{R} for 30 min at 4° C. The cells were either washed out or unchanged as indicated and replaced with media containing DMSO, MBX-4992, or heparin followed by incubation at 37° C. At 18 hpi, virus infection was evaluated using a Celigo cytometer.

As expected, MBX-4992 and heparin significantly inhibited virus infection under conditions in which the drugs were not removed or when added back to the washed-out cells. Yet, cells pre-incubated with MBX-4992 and sequentially removed do not significantly impact infection. These results imply that MBX-4992 does not significantly act upon a cellular factor to limit virus infection. Collectively, the virus and cell pre-incubation studies as shown in FIGS. 5B and 5C) demonstrate that MBX-4992 is a functional inhibitor only when incubated with the virus during the course of infection.

### Example 7. Analysis of NAPA Analogs

NAPA variants with modified groups were evaluated for inhibition of CMV infection in fibroblasts. The results are shown in FIG. 6. AD169^{R} infected fibroblasts were treated with NAPA compounds MBX-4992, MBXC-4325, MBXC-4330, and MBXC-4336 (0-50 μM) and analyzed for infection at 24 hpi (FIG. 6A-D). The infected cells were probed for IE1 as an indication of virus infection.

Results showed there was a concentration dependent decrease of infection upon treatment with all compounds with low IC₅₀ values ranging from 2.9-10.7 μM (FIG. 6A-D). The NAPA analog MBXC-4336 (FIG. 6D) was the most effective compound at inhibiting a CMV infection with an IC₅₀ of 2.9 μM. The effectiveness of MBXC-4336 was validated using the TR, Towne, and TB40/E strains (FIG. 11F-H) and upon immunoblot analysis (FIG. 12). These data support the model that NAPA analogs are effective CMV inhibitors and modification of the R₁ side chain to a larger aromatic ring seems to enhance the inhibitory characteristics of the compound.

### Example 8. Evaluation of Cytotoxicity of NAPA Compounds MBX-4992 and MBXC-4336

Next, the cytostaticity and cytotoxicity of treatment with MBX-4992 and MBXC-4336 was analyzed. The results are shown in FIG. 7A-D. NHDF cells treated with MBX-4992 and MBXC-4336 (0-50 μM) or cycloheximide (CHX) (50 μg/mL) for up to 6 days were analyzed for cell number by staining with Hoechst reagent (FIG. 7A-B) or ATP levels using CellTiter Glo luciferase activity assay (FIG. 7C-D).

Results show that treatment did not significantly reduce cell number over a 6 day period, even when the compounds were used at the highest concentration of 50 μM, indicating that the NAPA compounds do not impact cell proliferation. Upon examination of ATP levels following long term MBX-4992 treatment, no decrease in luciferase signal at either time point was seen (FIG. 7C). A small (10%) decrease of luciferase activity in MBXC-4336 treated cells was seen at Day 6 (FIG. 7D). As expected, cycloheximide treated cells demonstrated a dramatic decrease in cell number and luciferase activity (FIG. 7A-D). Collectively, these results imply that MBX-4992 and MBXC-4336 advantageously do not induce cell toxicity or limit cell proliferation at concentrations that limit virus infection (2.8-8.6 μM).

### Example 9. NAPA Compounds Limit CMV Dissemination

Previous studies conducted in our laboratory demonstrated that the NAPA compounds were effective at limiting virus proliferation in fibroblasts. To address whether NAPA compounds can limit virus dissemination in epithelial cells, we analyzed the inhibitory properties of MBX-4992 and MBXC-4336 using a plaque reduction assay by designating a cluster of virus infected cells as a virus plaque (Parsons et al., Antivir. Res., 193:105124 (2021)) The results are shown in FIG. 8A-H.

To evaluate the prophylactic function of the NAPA compounds, ARPE-19 cells and NHDF cells treated with DMSO, MBX-4992, MBXC-4336 (0-50 μM), or ganciclovir (2.5 μM) were infected with AD169^{R} CMV virus and analyzed for virus plaques at 10 dpi based on GFP fluorescence (FIG. 8A-F). A cluster of virus infected cells was designated a viral plaque based on an average cluster area of 5,000 μm² or 10,000 μm² (FIG. 8A-B) using a Celigo cytometer.

As seen in FIG. 8, both MBX-4992 and MBXC-4336 treatment significantly reduced virus plaques at 10 dpi in a concentration dependent manner with IC50 values of 6.5 μM and 12 μM for MBX-4992 and 1.7 μM and 4 μM for MBXC-4336 in both ARPE-19 cells and fibroblasts, respectively (FIG. 8C-F). As expected, ganciclovir treatment reduced plaque number by ˜50%. Further, MBX-4992 treatment significantly decreased generation of infectious AD169^{R} virions from MRC5 fibroblasts and ARPE-19 cells (FIGS. 13A & B). These results demonstrate that limiting virus infection by NAPA treatment significantly decreases virus dissemination and proliferation.

To evaluate the therapeutic function of the NAPA compounds, we examined whether MBX-4992 and MBXC-4336 can therapeutically limit virus dissemination in CMV-infected cells. The results are shown in FIG. 8G-H. AD169^{R}-CMV virus infected ARPE-19 cells treated at 48 hpi with DMSO, MBX-4992, MBXC-4336 (0-50 μM), or ganciclovir (2.5 μM) were analyzed in a plaque reduction assay using a Celigo cytometer.

Both MBX-4992 and MBXC-4336 reduced plaques >5,000 μm² and >10,000 μm² with IC₅₀˜9 μM of the AD169^{R}-infected cells. These findings further demonstrate that MBX-4992 and MBXC-4336 limit virus dissemination with similar efficacy as ganciclovir. Together, the results indicate that the NAPA compounds are able to efficiently limit virus dissemination when used in a prophylactic and therapeutic in vitro setting.

### Example 10. MBX-4992 and Ganciclovir Combination Inhibits CMV Dissemination

Ganciclovir is a nucleoside analog that inhibits the late stage of the CMV life cycle by targeting the viral polymerase UL54 protein (Biron et al., Antivir. Res., 71:154-63 (2006)). In contrast, as demonstrated previously, the NAPA compounds appear to target an early step of infection (see, FIG. 5). We next examined whether MBX-4992 in combination with ganciclovir can exhibit a synergistic effect to limit virus dissemination. The results are shown in FIG. 9A-D. The plaques were examined for TB40/E-infected ARPE-19 and NHDF cells treated with the combination of ganciclovir (2.5u M) and increasing concentrations of MBX-4992 (1-20 μM) at 10 dpi based on GFP fluorescence (FIG. 9A-B).

The viral plaques, based on a viral cluster area >10,000 μm², decreased upon treatment with MBX-4992 or ganciclovir. Quantification of the plaques demonstrated that ARPE-19 cells treated with MBX-4992 and ganciclovir advantageously resulted in an enhanced decrease in viral plaques when compared to ganciclovir or MBX-4992 alone (FIGS. 9A and C). Note that the plaque numbers in the untreated cells was much higher than reported due to the spread of virus causing large regions of virus infection (FIGS. 9A and B). In contrast, the combination of MBX-4992 and ganciclovir treatment in fibroblasts did not cause an enhanced decrease in viral plaques when compared to mono-drug treatment (FIGS. 9B and D). At 10 days post-infection, representative whole well images of plaques >10,000 μm² (A and B) were quantified based on GFP fluorescence intensity and calculated relative to DMSO controls (C and D).

These findings indicate that MBX-4992 advantageously works synergistically with ganciclovir to prevent virus dissemination in epithelial cells. The results support the paradigm that the drug combination of a NAPA compound and ganciclovir is more effective at inhibiting viral dissemination in epithelial cells suggesting the drugs complement their function to enhance viral inhibition.

1-16. (canceled)

17. A compound for treating or preventing cytomegalovirus infection in a mammalian subject, said compound comprising Formula I: 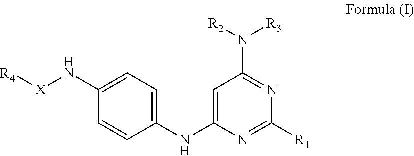
wherein:
R₁ is selected from a hydrogen atom, or a C1-C3 alkyl group;
R₂ and R₃ are selected from a hydrogen atom, a C1-C3 alkyl group, an aromatic ring with 1-3 substituents selected from a halogen, a C1-C4 alkyl group, a C1-C4 alkoxy group, or a C1-C4 amino-alkyl group, a heteroaromatic ring with 1-3 substituents selected from a halogen, C1-C4 alkyl group, a C1-C4 alkoxy group, or a C1-C4 amino-alkyl group,
R₂ and R₃ may be a bridging chain to form a heterocyclic ring with 1-3 substituents selected from a C1-C4 alkyl group, a C1-C4 alkoxy group, or a C1-C4 amino-alkyl group,
R₄ is a substituted or non-substituted furan, thiophene, or phenyl ring; and
X is either a carbonyl (CO) group or a sulfonyl (SO₂) group;
or a pharmaceutically acceptable salt thereof.

18. The compound according to claim 17 further comprising a pharmaceutically acceptable carrier or excipient.

19. The compound according to claim 17, wherein said pharmaceutically acceptable carrier or excipient further comprises at least one additional antiviral agent or compound.

20. A pharmaceutical composition comprising one or more compounds according to claim 17 and a pharmaceutically acceptable carrier or excipient.

21. The compound according to claim 17 formulated for oral, parenteral, or topical administration.

22. A compound for treating or preventing cytomegalovirus infection in a mammalian subject, said compound comprising Formula II: 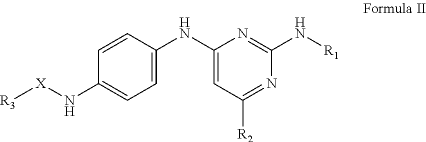
wherein:
R₁ is a substituted phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is a substituted phenyl or furan ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is either a carbonyl (CO) group, a sulfonyl (SO₂) group, or a NHCO group (part of a urea).

23. The compound according to claim 22 further comprising a pharmaceutically acceptable carrier or excipient.

24. The compound according to claim 22, wherein said pharmaceutically acceptable carrier or excipient further comprises at least one additional antiviral agent or compound.

25. A pharmaceutical composition comprising one or more compounds according to claim 22 and a pharmaceutically acceptable carrier or excipient.

26. The compound according to claim 22 formulated for oral, parenteral, or topical administration.

27. A compound for treating or preventing cytomegalovirus infection in a mammalian subject, said compound comprising Formula III: 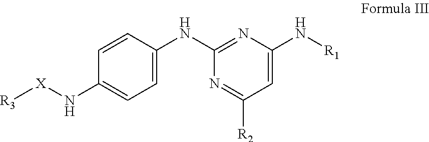
wherein:
R₁ is a phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is substituted phenyl ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is a carbonyl (CO) group.

28. The compound according to claim 27 further comprising a pharmaceutically acceptable carrier or excipient.

29. The compound according to claim 27, wherein said pharmaceutically acceptable carrier or excipient further comprises at least one additional antiviral agent or compound.

30. A pharmaceutical composition comprising one or more compounds according to claim 27 and a pharmaceutically acceptable carrier or excipient.

31. The compound according to claim 27 formulated for oral, parenteral, or topical administration.

32. The cytomegalovirus inhibitor compound according to Formula I, Formula II, or Formula III, wherein said compound is selected from the group consisting of:

| Cmpd # | Structure |
|---|---|
| MBXC- 4294 | 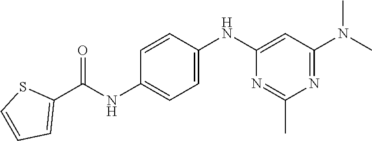 [SMILES: Cc1nc(Nc2ccc(NC(=O)c3cccs3)cc2)cc(N(C)C)n1] |
| MBXC- 4295 | 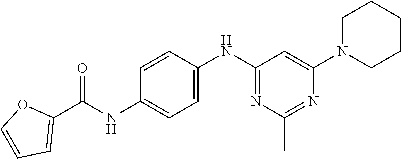 [SMILES: Cc1nc(Nc2ccc(NC(=O)c3ccco3)cc2)cc(N2CCCCC2)n1] |
| MBXC- 4297 | 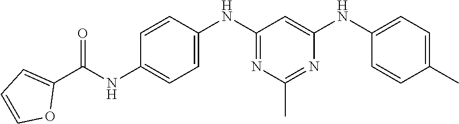 [SMILES: Cc1ccc(Nc2cc(Nc3ccc(NC(=O)c4ccco4)cc3)nc(C)n2)cc1] |
| MBXC- 4298 | 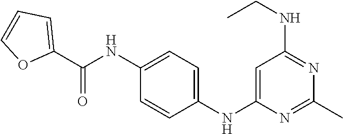 [SMILES: CCNc1cc(Nc2ccc(NC(=O)c3ccco3)cc2)nc(C)n1] |
| MBXC- 4299 | 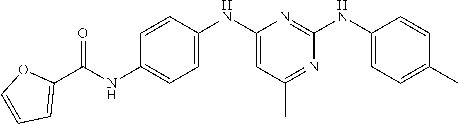 [SMILES: Cc1ccc(Nc2nc(C)cc(Nc3ccc(NC(=O)c4ccco4)cc3)n2)cc1] |
| MBXC- 4300 | 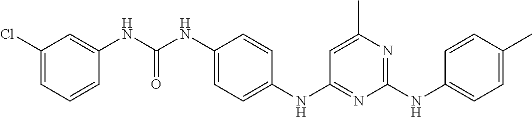 [SMILES: Cc1ccc(Nc2nc(C)cc(Nc3ccc(NC(=O)Nc4cccc(Cl)c4)cc3)n2)cc1] |
| MBXC- 4301 | 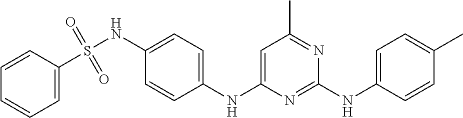 [SMILES: Cc1ccc(Nc2nc(C)cc(Nc3ccc(NS(=O)(=O)c4ccccc4)cc3)n2)cc1] |
| MBXC- 4302 | 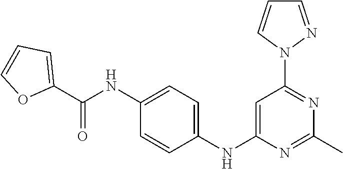 [SMILES: Cc1nc(Nc2ccc(NC(=O)c3ccco3)cc2)cc(-n2cccn2)n1] |
| MBXC- 4303 | 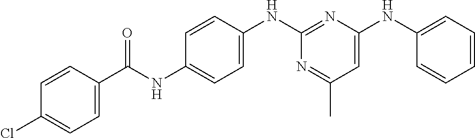 [SMILES: Cc1cc(Nc2ccccc2)nc(Nc2ccc(NC(=O)c3ccc(Cl)cc3)cc2)n1] |
| MBXC- 4304 | 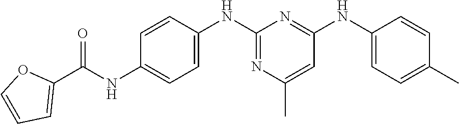 [SMILES: Cc1ccc(Nc2cc(C)nc(Nc3ccc(NC(=O)c4ccco4)cc3)n2)cc1] |
| MBXC- 4325 | 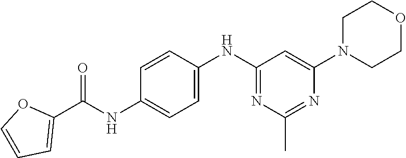 [SMILES: Cc1nc(Nc2ccc(NC(=O)c3ccco3)cc2)cc(N2CCOCC2)n1] |
| MBXC- 4330 | 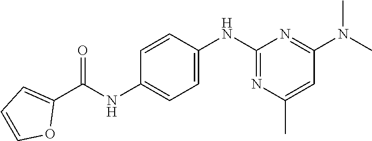 [SMILES: Cc1cc(N(C)C)nc(Nc2ccc(NC(=O)c3ccco3)cc2)n1] |
| MBXC- 4336 | 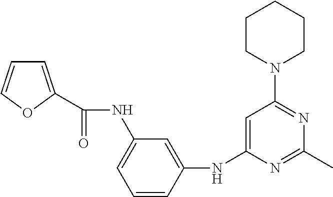 [SMILES: Cc1nc(Nc2cccc(NC(=O)c3ccco3)c2)cc(N2CCCCC2)n1] |
| MBX- 4991 | 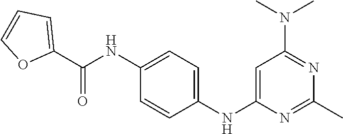 [SMILES: Cc1nc(Nc2ccc(NC(=O)c3ccco3)cc2)cc(N(C)C)n1] |
| MBX- 4992 | 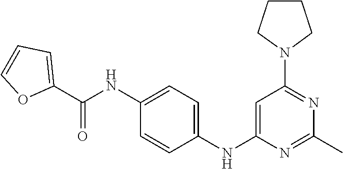 [SMILES: Cc1nc(Nc2ccc(NC(=O)c3ccco3)cc2)cc(N2CCCC2)n1] |

33. A method for inhibiting cytomegalovirus infection in a mammal comprising administering to a mammal in need thereof an effective amount of a composition comprising a compound of Formula I, 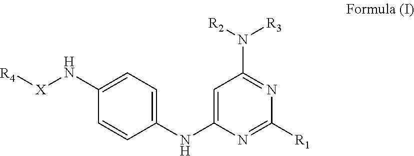
wherein:
R₁ is selected from a hydrogen atom, or a C1-C3 alkyl group;
R₂ and R₃ are selected from a hydrogen atom, a C1-C3 alkyl group, an aromatic ring with 1-3 substituents selected from a halogen, a C1-C4 alkyl group, a C1-C4 alkoxy group, or a C1-C4 amino-alkyl group, a heteroaromatic ring with 1-3 substituents selected from a halogen, C1-C4 alkyl group, a C1-C4 alkoxy group, or a C1-C4 amino-alkyl group,
R₂ and R₃ may be a bridging chain to form a heterocyclic ring with 1-3 substituents selected from a C1-C4 alkyl group, a C1-C4 alkoxy group, or a C1-C4 amino-alkyl group,
R₄ is a substituted or non-substituted furan, thiophene, or phenyl ring; and
X is either a carbonyl (CO) group or a sulfonyl (SO₂) group;
or a pharmaceutically acceptable salt thereof.

34. The method according to claim 33, wherein the composition is formulated for administration in a pharmaceutically acceptable carrier or excipient.

35. The method according to claim 33, wherein the mammal is a human.

36. The method according to claim 33, wherein the composition further comprises a second antiviral agent.

37. A method for inhibiting cytomegalovirus infection in a mammal comprising administering to a mammal in need thereof an effective amount of a composition comprising a compound of Formula II 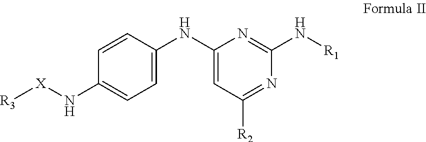
wherein:
R₁ is a substituted phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is a substituted phenyl or furan ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is either a carbonyl (CO) group, a sulfonyl (SO₂) group, or a NHCO group (part of a urea).

38. The method according to claim 37, wherein the composition is formulated for administration in a pharmaceutically acceptable carrier or excipient.

39. The method according to claim 37, wherein the mammal is a human.

40. The method according to claim 37, wherein the composition further comprises a second antiviral agent.

41. A method for inhibiting cytomegalovirus infection in a mammal comprising administering to a mammal in need thereof an effective amount of a composition comprising a compound of Formula III, 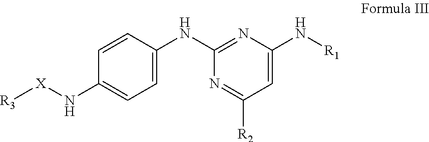
wherein:
R₁ is a phenyl ring with 1-3 substituents selected from a halogen, C1-C4 alkyl group, a C1-C4 alkoxy group, or a C1-C4 amino-alkyl group;
R₂ is a C1-C3 alkyl group;
R₃ is substituted phenyl ring with 1-3 substituents selected from a halogen, C1-C4 alkyl group, a C1-C4 alkoxy group, or a C1-C4 amino-alkyl group, or a substituted furan ring with 1-3 substituents selected from a C1-C4 alkyl group, a C1-C4 alkoxy group, or a C1-C4 amino-alkyl group; and
X is a carbonyl (CO) group.

42. The method according to claim 41, wherein the composition is formulated for administration in a pharmaceutically acceptable carrier or excipient.

43. The method according to claim 41, wherein the mammal is a human.

44. The method according to claim 41, wherein the composition further comprises a second antiviral agent.

45. A method for inhibiting cytomegalovirus infection in a mammal comprising administering to a mammal in need thereof an effective amount of a composition comprising a compound of Formula I, Formula II, or Formula III, wherein said compound is selected from the group consisting of:

| Cmpd # | Structure |
|---|---|
| MBXC- 4294 | 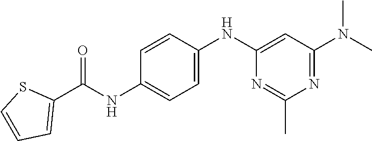 [SMILES: Cc1nc(Nc2ccc(NC(=O)c3cccs3)cc2)cc(N(C)C)n1] |
| MBXC- 4295 | 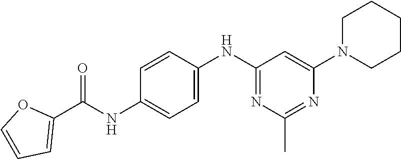 [SMILES: Cc1nc(Nc2ccc(NC(=O)c3ccco3)cc2)cc(N2CCCCC2)n1] |
| MBXC- 4297 | 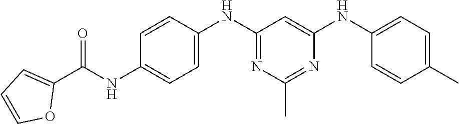 [SMILES: Cc1ccc(Nc2cc(Nc3ccc(NC(=O)c4ccco4)cc3)nc(C)n2)cc1] |
| MBXC- 4298 | 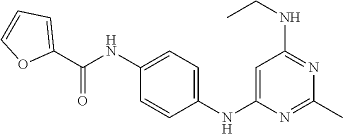 [SMILES: CCNc1cc(Nc2ccc(NC(=O)c3ccco3)cc2)nc(C)n1] |
| MBXC- 4299 | 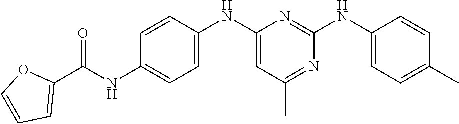 [SMILES: Cc1ccc(Nc2nc(C)cc(Nc3ccc(NC(=O)c4ccco4)cc3)n2)cc1] |
| MBXC- 4300 | 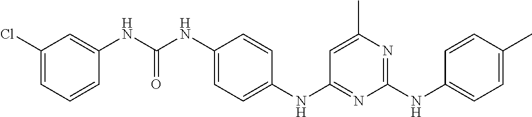 [SMILES: Cc1ccc(Nc2nc(C)cc(Nc3ccc(NC(=O)Nc4cccc(Cl)c4)cc3)n2)cc1] |
| MBXC- 4301 | 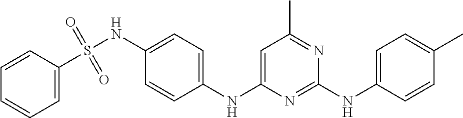 [SMILES: Cc1ccc(Nc2nc(C)cc(Nc3ccc(NS(=O)(=O)c4ccccc4)cc3)n2)cc1] |
| MBXC- 4302 | 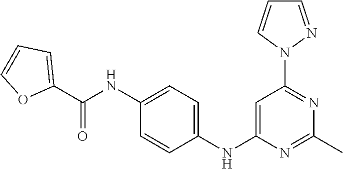 [SMILES: Cc1nc(Nc2ccc(NC(=O)c3ccco3)cc2)cc(-n2cccn2)n1] |
| MBXC- 4303 | 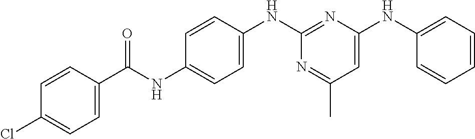 [SMILES: Cc1cc(Nc2ccccc2)nc(Nc2ccc(NC(=O)c3ccc(Cl)cc3)cc2)n1] |
| MBXC- 4304 | 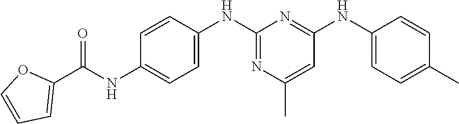 [SMILES: Cc1ccc(Nc2cc(C)nc(Nc3ccc(NC(=O)c4ccco4)cc3)n2)cc1] |
| MBXC- 4325 | 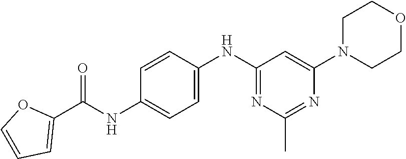 [SMILES: Cc1nc(Nc2ccc(NC(=O)c3ccco3)cc2)cc(N2CCOCC2)n1] |
| MBXC- 4330 | 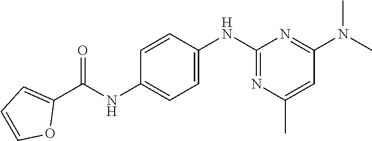 [SMILES: Cc1cc(N(C)C)nc(Nc2ccc(NC(=O)c3ccco3)cc2)n1] |
| MBXC- 4336 | 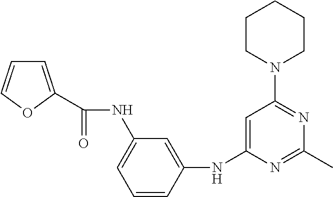 [SMILES: Cc1nc(Nc2cccc(NC(=O)c3ccco3)c2)cc(N2CCCCC2)n1] |
| MBX- 4991 | 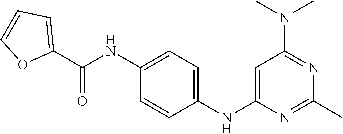 [SMILES: Cc1nc(Nc2ccc(NC(=O)c3ccco3)cc2)cc(N(C)C)n1] |
| MBX- 4992 | 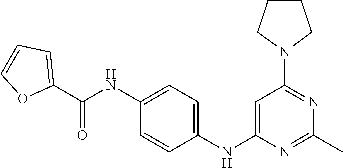 [SMILES: Cc1nc(Nc2ccc(NC(=O)c3ccco3)cc2)cc(N2CCCC2)n1] |
